(19) United States
(12) Patent Application Publication
(10) Pub. No.: US 2021/0000874 A1
(43) Pub. Date: Jan. 7, 2021

(54) METHODS OF SELECTING T CELL LINE FOR ADOPTIVE CELLULAR THERAPY

(71) Applicant: Memorial Sloan Kettering Cancer Center, New York, NY (US)

(72) Inventors: Richard John O'Reilly, Roxbury, CT (US); Ekaterina Doubrovina, Bronx, NY (US); Susan Elizabeth Prockop, New York, NY (US)

(73) Assignee: Memorial Sloan Kettering Cancer Center, New York, NY (US)

(21) Appl. No.: 16/979,396

(22) PCT Filed: Mar. 13, 2019
(86) PCT No.: PCT/US2019/021961

§ 371 Date: Sep. 9, 2020

Related U.S. Application Data
(60) Provisional application No. 62/642,970, filed on Mar. 14, 2018.

(51) Int. Cl. A61K 35/17; G01N 33/50; A61P 35/00

(52) U.S. Cl. CPC A61K 35/17; A61P 35/00; G01N 33/505

(57) ABSTRACT

Provided herein are methods of selecting a T cell line from among a collection of T cell lines for therapeutic administration to a human patient to treat a disease or disorder associated with a pathogen or to treat a cancer in the human patient. When the human patient has not been the recipient of any cellular transplant, the method involves excluding T cell lines restricted by only one HLA allele shared with the human patient and selecting a T cell line that is restricted to more than one HLA allele shared with the human patient and that exhibits a T cell response against an antigen of the pathogen or cancer. When the human patient has been the recipient of a cellular transplant, the method involves excluding T cell lines restricted by only one HLA allele shared with an entity selected from the group consisting of (i) the diseased cells in the human patient that express the one or more antigens of the pathogen or cancer, (ii) the human patient, (iii) the donor of the cellular transplant, and (iv) both the human patient and the donor of the cellular transplant, and selecting a T cell line that is restricted to more than one HLA allele shared with the entity and that exhibits a T cell response against an antigen of the pathogen or cancer.

### CROSS-REFERENCE TO RELATED APPLICATIONS

This application claims the benefit of U.S. Provisional Application No. 62/642,970, filed on Mar. 14, 2018, which is incorporated by reference herein in its entirety.

### GOVERNMENT RIGHTS STATEMENT

This invention was made with government support under CA162002 and CA023766 awarded by National Institutes of Health. The Government has certain rights in the invention.

### 1. FIELD

Provided herein are methods of selecting a T cell line from among a collection of T cell lines for therapeutic administration to a human patient to treat a disease or disorder associated with a pathogen or to treat a cancer in the human patient. When the human patient has not been the recipient of any cellular transplant, the method involves excluding T cell lines restricted by only one HLA allele shared with the human patient and selecting a T cell line that is restricted to more than one HLA allele shared with the human patient and that exhibits a T cell response against an antigen of the pathogen or cancer. When the human patient has been the recipient of a cellular transplant, the method involves excluding T cell lines restricted by only one HLA allele shared with an entity selected from the group consisting of (i) the diseased cells in the human patient that express the one or more antigens of the pathogen or cancer, (ii) the human patient, (iii) the donor of the cellular transplant, and (iv) both the human patient and the donor of the cellular transplant, and selecting a T cell line that is restricted to more than one HLA allele shared with the entity and that exhibits a T cell response against an antigen of the pathogen or cancer.

### 2. BACKGROUND

Antigen-specific T cells can be used in adoptive immunotherapy to treat infections and cancer, such as cytomegalovirus (CMV) infections, Epstein-Barr virus-associated lymphoproliferative disorder (EBV-LPD) and EBV-positive nasopharyngeal carcinoma, and WT1 (Wilms Tumor 1)-positive leukemia and multiple myeloma (see, e.g., Prockop et al., 2016, J Clin Oncol 34:3012; Koehne et al., 2015, Blood 126:98; Koehne et al., 2015, Biol Blood Marrow Transplant 21:1663-1678; O'Reilly et al., 2012, Seminars in Immunology 22:162-172; Doubrovina et al., 2012, Blood 119:2644-2656; and Barker et al., 2010, Blood 116:5045-5049). There is a need for methods of selecting T cell lines to improve efficacy of treatment.

Citation of a reference herein shall not be construed as an admission that such is prior art to the present disclosure.

### 3. SUMMARY OF THE INVENTION

In one aspect, provided herein is a method of selecting a T cell line from among a collection of T cell lines for therapeutic administration to a human patient to treat a disease or disorder associated with a pathogen or to treat a cancer in the human patient, wherein the human patient has not been the recipient of any cellular transplant, said method comprising: (a) identifying those T cell lines in the collection that exhibit a T cell response against one or more antigens of the pathogen or cancer and that are restricted by one or more HLA alleles shared with the human patient; (b) excluding from the T cell lines identified in step (a) those T cell lines that exhibit a T cell response against said one or more antigens of the pathogen or cancer and that are restricted by only one HLA allele shared with the human patient; and (c) selecting for therapeutic administration to said human patient a T cell line from among those identified T cell lines remaining after step (b). In a specific embodiment, the method of selecting a T cell line further comprises before step (a) a step of ascertaining the HLA restriction of each T cell line in the collection. In a specific embodiment, the method of selecting a T cell line further comprises before step (a) a step of ascertaining the HLA assignment of the human patient. In a further specific embodiment, the step of ascertaining the HLA assignment of the human patient comprises typing at least 4 HLA loci. In a specific embodiment, the selected T cell line is derived from a human donor that is allogeneic to the human patient.

In another aspect, provided herein is a method of selecting a T cell line from among a collection of T cell lines for therapeutic administration to a human patient to treat a disease or disorder associated with a pathogen or to treat a cancer in the human patient, wherein the human patient has been the recipient of a cellular transplant, said method comprising: (a) identifying those T cell lines in the collection that exhibit a T cell response against one or more antigens of the pathogen or cancer and that are restricted by one or more HLA alleles shared with an entity selected from the group consisting of (i) the diseased cells in the human patient that express the one or more antigens of the pathogen or cancer, (ii) the human patient, (iii) the donor of the cellular transplant, and (iv) both the human patient and the donor of the cellular transplant; (b) excluding from the T cell lines identified in step (a) those T cell lines that exhibit a T cell response against said one or more antigens of the pathogen or cancer and that are restricted by only one HLA allele shared with the entity; and (c) selecting for therapeutic administration to said human patient a T cell line from among those identified T cell lines remaining after step (b). The entity that is selected from the group consisting of (i) the diseased cells in the human patient that express the one or more antigens of the pathogen or cancer, (ii) the human patient, (iii) the donor of the cellular transplant, and (iv) both the human patient and the donor of the cellular transplant; in such a method shall be referred to herein as the “Entity,” for purposes of convenience. In a specific embodiment, the method of selecting a T cell line further comprises before step (a) a step of ascertaining the HLA restriction of each T cell line in the collection. In a specific embodiment, the method of selecting a T cell line further comprises before step (a) a step of ascertaining the HLA assignment of the Entity. In a further specific embodiment, the step of ascertaining the HLA assignment of the Entity comprises typing at least 4 HLA loci. In a specific embodiment, the selected T cell line is derived from a human donor that is allogeneic to the human patient. In a further specific embodiment, the human donor is a third-party donor that is different from the donor of the cellular transplant. In some embodiments, the cellular transplant is a hematopoietic stem cell transplant (HSCT). In a specific embodiment, the cellular transplant is HSCT, the disease or disorder or the cancer is an EBV-associated post-transplant lymphoproliferative disorder (EBV-PTLD), and the Entity is the donor of the cellular transplant. In other embodiments, the cellular transplant is a solid organ transplant (SOT) (for example, a kidney transplant, a liver transplant, a heart transplant, an intestinal transplant, a pancreas transplant, a lung transplant, or a small bowel transplant). In a specific embodiment, the cellular transplant is an SOT (for example, a kidney transplant, a liver transplant, a heart transplant, an intestinal transplant, a pancreas transplant, a lung transplant, or a small bowel transplant), the disease or disorder or the cancer is an EBV-PTLD, and the Entity is the human patient.

In some embodiments, the method of selecting a T cell line described herein is of selecting a T cell line for therapeutic administration to the human patient to treat a disease or disorder associated with a pathogen in the human patient, and the one or more antigens are one or more antigens of the pathogen. In certain embodiments, the pathogen is a virus, bacterium, fungus, helminth or protist. In specific embodiments, the pathogen is a virus. In a specific embodiment, the virus is cytomegalovirus (CMV) (for example, when the disease or disorder is CMV infection). In an aspect of the specific embodiment, the one or more antigens are CMV pp65, CMV IE1, or a combination thereof. In another specific embodiment, the virus is Epstein-Barr virus (EBV). In an aspect of the specific embodiment, the one or more antigens are EBNA1, EBNA2, EBNA3A, EBNA3B, EBNA3C, LMP1, LMP2, or a combination thereof. In another specific embodiment, the virus is BK virus (BKV), John Cunningham virus (JCV), human herpesvirus, human papillomavirus (HPV), hepatitis B virus (HBV), hepatitis C virus (HCV), herpes simplex virus (HSV), varicella zoster virus (VZV), Merkel cell polyomavirus (MCV), adenovirus (ADV), human immunodeficiency virus (HIV), influenza virus, ebola virus, poxvirus, rhabdovirus, or paramyxovirus.

In other embodiments, the method of selecting a T cell line described herein is of selecting a T cell line for therapeutic administration to the human patient to treat a cancer in the human patient, and the one or more antigens are one or more antigens of the cancer. In a specific embodiment, the cancer is a blood cancer. In another specific embodiment, the cancer is a cancer of the breast, lung, ovary, stomach, pancreas, larynx, esophagus, testes, liver, parotid, biliary tract, colon, rectum, cervix, uterus, endometrium, kidney, bladder, prostate, thyroid, brain or skin. In a specific embodiment, the one or more antigens is Wilms Tumor 1 (WT1) (for example, when the cancer is multiple myeloma or plasma cell leukemia). In another specific embodiment, the one or more antigens are one or more antigens of EBV (for example, when the cancer is an EBV-positive lymphoproliferative disorder). In another specific embodiment, the one or more antigens are one or more antigens of CMV (for example, when the cancer is CMV-positive glioblastoma multiforme).

### 4. BRIEF DESCRIPTION OF FIGURE

FIGS. 1A-1C. Characterization of EBV-specific cytotoxic T cells infused. (A) Phenotype [CD3 (circle), CD8 (square), CD4 (triangle) and NK (triangle)]. (B) Cytotoxic activity of EBV-specific T cell lines against autologous BLCLs (circle), autologous PHA blasts (square); mismatched targets (triangle) and NK sensitive K562 targets (triangle). (C) EBV-CTL precursor frequency (circle) and alloreactive CTL precursor frequency (circles) in lines infused to treat patients.

FIG. 2. Number of cycles to best response (CR or PR). Cumulative responses to successive cycles of EBV-CTLs following first T cell line ultimately responsible for inducing a partial or complete remission. The percent of patients achieving a complete response (CR) in black and partial response (PR) in gray after each cycle of EBV-CTLs.

FIGS. 3A-3B. Overall responses of EBV lymphomas to treatment. (A) Table summarizing best responses by treatment cohort (HCT or SOT). (B) Kaplan-Meier curve of overall survival at 1 year by type of transplant HCT (the top line) and SOT (the bottom line) recipients treated with HLA partially-matched EBV-specific CTLs restricted by a shared HLA allele.

FIG. 4. Flow chart of treatment and responses for patients treated for EBV-PTLD with 3^{rd} party EBV-CTLs. Flow chart of treatment and responses for patients treated on protocol for EBV-PTLD with third party EBV-CTLs. CR=complete response, RP=partial response, SD=stable disease, POD=progression of disease, DOD=dead of disease.

FIGS. 5A-5B. Overall survival at 1 year based on response to first cycle of EBV-CTLs. (A) Overall survival at 1 year of responding patients (the top line), patients with stable disease (the middle line) and those with POD (the bottom line). (B) Overall survival at 1 year of patients experiencing POD after the first cycle of EBV-CTLs based on whether additional cycles of EBV-CTLs from a different donor were administered (the top line) or no further therapy was administered (the bottom line).

FIGS. 6A-6C. EBV-specific CTL precursor frequency measured by limiting dilution analysis and compared by Student two-tailed T test in patients prior to and subsequent to receiving EBV-CTL therapy. (A) Baseline precursor frequency measured in patients who went on to either respond (gray bar) or not (black bar) to EBV-CTL therapy. (B) Peak EBV-CTL precursor frequency in responding (gray bar) and non-responding (black bar) patients. (C) EBV-CTL precursor frequency in responding HCT (gray bar) versus SOT (pale gray bar) recipients.

FIG. 7. EBV-specific CTL precursor frequency does not predict response. EBV-specific CTL precursor frequency measured by limiting dilution analysis in EBV-CTL lines used to treat alloHCT and alloSOT patients. As compared by Mann Whitney no significant differences were observed between lines producing responses (circles) and those that failed (squares).

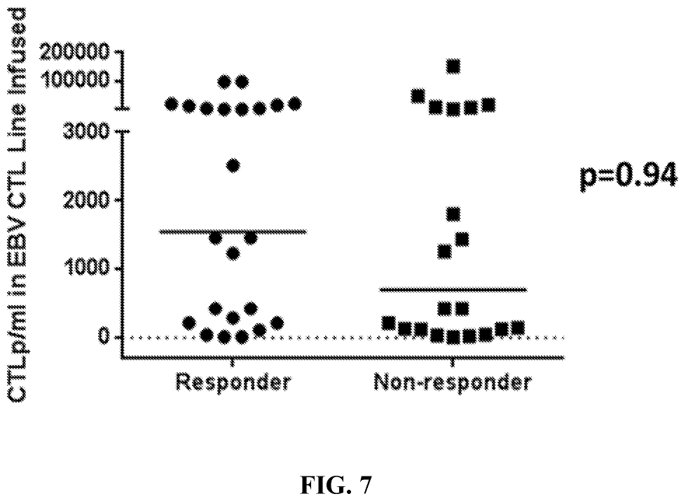

FIG. 8. EBV-CTLp frequency after 1^{st} cycle of adoptive therapy with 3^{rd} party EBV-CTLs. Expansions could be detected in patients with responses as well as those with stable disease. Individual patients demonstrated in different lines.

FIGS. 9A-9D. Response to EBV-CTLs restricted by either HLA A*1101 or HLA B*4403. (A) High resolution typing of EBV+ lymphoma derived from the non-engrafting cord blood unit and of the four EBV-CTL lines successively infused. The underlined HLA alleles indicate the restricting HLA allele(s) of the EBV-CTL line. (B) Time course of EBV lymphoma and response to successive EBV-CTL lines (LDH as a surrogate blood marker of disease in this patient who did not have detectable EBV DNA in the blood following rituximab. (C) Successive PET scans of disease progression and regression. (D) Cytolytic activity of the successive lines used against B95.8 transformed EBV-BLCLs of each T cell donor and against endogenous EBV transformants cultured from the biopsy proven lymphoma tissue.

### 5. DETAILED DESCRIPTION

The present invention provides methods of selecting a T cell line from among a collection of T cell lines for therapeutic administration to a human patient to treat a disease or disorder associated with a pathogen or to treat a cancer in the human patient.

5.1. Selection of T Cell Line

In one aspect, provided herein is a method of selecting a T cell line from among a collection of T cell lines for therapeutic administration to a human patient to treat a disease or disorder associated with a pathogen or to treat a cancer in the human patient, wherein the human patient has not been the recipient of any cellular transplant, said method comprising: (a) identifying those T cell lines in the collection that exhibit a T cell response against one or more antigens of the pathogen or cancer and that are restricted by one or more HLA alleles shared with the human patient; (b) excluding from the T cell lines identified in step (a) those T cell lines that exhibit a T cell response against said one or more antigens of the pathogen or cancer and that are restricted by only one HLA allele shared with the human patient; and (c) selecting for therapeutic administration to said human patient a T cell line from among those identified T cell lines remaining after step (b). In a specific embodiment, the method of selecting a T cell line further comprises before step (a) a step of ascertaining the HLA restriction of each T cell line in the collection. In a specific embodiment, the method of selecting a T cell line further comprises before step (a) a step of ascertaining the HLA assignment of the human patient (see Section 5.1.1.3, infra, for more details). In a further specific embodiment, the step of ascertaining the HLA assignment of the human patient comprises typing at least 4 HLA loci. In a specific embodiment, the method of selecting a T cell line further comprises before step (a) a step of ascertaining the HLA restriction of each T cell line in the collection (see Section 5.1.1.3, infra, for more details). In a specific embodiment, the selected T cell line is derived from a human donor that is allogeneic to the human patient.

In another aspect, provided herein is a method of selecting a T cell line from among a collection of T cell lines for therapeutic administration to a human patient to treat a disease or disorder associated with a pathogen or to treat a cancer in the human patient, wherein the human patient has been the recipient of a cellular transplant, said method comprising: (a) identifying those T cell lines in the collection that exhibit a T cell response against one or more antigens of the pathogen or cancer and that are restricted by one or more HLA alleles shared with an entity selected from the group consisting of (i) the diseased cells in the human patient that express the one or more antigens of the pathogen or cancer, (ii) the human patient, (iii) the donor of the cellular transplant, and (iv) both the human patient and the donor of the cellular transplant; (b) excluding from the T cell lines identified in step (a) those T cell lines that exhibit a T cell response against said one or more antigens of the pathogen or cancer and that are restricted by only one HLA allele shared with the entity; and (c) selecting for therapeutic administration to said human patient a T cell line from among those identified T cell lines remaining after step (b). The entity that is selected from the group consisting of (i) the diseased cells in the human patient that express the one or more antigens of the pathogen or cancer, (ii) the human patient, (iii) the donor of the cellular transplant, and (iv) both the human patient and the donor of the cellular transplant; in such a method shall be referred to herein as the “Entity,” for purposes of convenience. In a specific embodiment, the method of selecting a T cell line further comprises before step (a) a step of ascertaining the HLA restriction of each T cell line in the collection. In a specific embodiment, the method of selecting a T cell line further comprises before step (a) a step of ascertaining the HLA assignment of the Entity (see Section 5.1.1.3, infra, for more details). In a further specific embodiment, the step of ascertaining the HLA assignment of the Entity comprises typing at least 4 HLA loci. In a specific embodiment, the method of selecting a T cell line further comprises before step (a) a step of ascertaining the HLA restriction of each T cell line in the collection (see Section 5.1.1.3, infra, for more details). In a specific embodiment, the selected T cell line is derived from a human donor that is allogeneic to the human patient. In a further specific embodiment, the human donor is a third-party donor that is different from the donor of the cellular transplant. In another further specific embodiment, the human donor is the donor of the cellular transplant.

In a specific embodiment, the selecting step (c) in a method of selecting a T cell line described herein is selecting for therapeutic administration to said human patient a T cell line that is restricted to the most number of HLA alleles shared with the human patient (when the human patient has not been the recipient of any cellular transplant) or the Entity (when the human patient has been the recipient of a cellular transplant) from among those identified T cell lines remaining after step (b).

A cellular transplant is a transplant comprising viable cells, including, for example, a hematopoietic stem cell transplant (HSCT) (such as a peripheral blood stem cell transplant, a bone marrow transplant, or a cord blood transplant), a tissue transplant (such as a skin transplant, a bone transplant, a tendon transplant, a cornea transplant, a heart valve transplant, a nerve transplant, or a vein transplant), or a solid organ transplant (SOT) (such as a kidney transplant, a liver transplant, a heart transplant, an intestinal transplant, a pancreas transplant, a lung transplant, or a small bowel transplant).

A “disease or disorder associated with a pathogen” as used herein refers to a disease or disorder that results from the presence of the pathogen, and can be, as non-limiting examples, a pathogen-positive cancer, viremia, or an infection by the pathogen.

5.1.1. Characteristics of T Cell Line

According to the invention, the T cell line selected according to a method described herein is restricted by more than one HLA allele shared with the human patient (when the human patient has not been the recipient of any cellular transplant) or the Entity (when the human patient has been the recipient of a cellular transplant) and exhibits a T cell response against one or more antigens of the pathogen or cancer.

In a specific embodiment, a T cell line (either the selected T cell line or a non-selected T cell line in the collection of T cell lines) exhibits a T cell response against one or more antigens of the pathogen or cancer if it exhibits substantial antigen reactivity (for example, cytotoxicity) in vitro toward fully or partially HLA-matched (relative to the T cell line) target antigen presenting cells that present the one or more antigens of the pathogen or cancer (e.g., target antigen presenting cells that are loaded with or genetically engineered to express one or more peptides or proteins derived from the one or more antigens of the pathogen or cancer), determined as described in Section 5.1.1.1, infra.

In another specific embodiment, a T cell line (either the selected T cell line or a non-selected T cell line in the collection of T cell lines) exhibits a T cell response against one or more antigens of the pathogen or cancer if it exhibits in vivo clinical efficacy in treatment of a disease or disorder associated with the pathogen or treatment of the cancer, for example, if at least one human patient whose disease or disorder associated with the pathogen or whose cancer (as the case may be) has achieved a complete remission (CR) or partial remission (PR) after treatment with the T cell line.

The HLA restriction of a T cell line (either the selected T cell line or a non-selected T cell line in the collection of T cell lines) can be ascertained as described in Section 5.1.1.3, infra. In a specific embodiment, the method of selecting a T cell line described herein further comprises before step (a) a step of ascertaining the HLA restriction of each T cell line in the collection.

In a specific embodiment, the method of selecting a T cell line further comprises before step (a) a step of ascertaining the HLA assignment of the human patient (when the human patient has not been the recipient of any cellular transplant) or the Entity (when the human patient has been the recipient of a cellular transplant). In various embodiments, the step of ascertaining comprises typing at least 4 HLA loci (preferably at least HLA-A, HLA-B, HLA-C, and HLA-DR (preferably HLA-DRB1)). In one embodiment, the step of ascertaining comprises typing 4 HLA loci (preferably HLA-A, HLA-B, HLA-C, and HLA-DR (preferably HLA-DRB1)). In another embodiment, the step of ascertaining comprises typing 5 HLA loci (preferably HLA-A, HLA-B, HLA-C, HLA-DR (preferably HLA-DRB1), and HLA-DQ (preferably HLA-DQB1)). In another embodiment, the step of ascertaining comprises typing 6 HLA loci. In another embodiment, the step of ascertaining comprises typing 7 HLA loci. In another embodiment, the step of ascertaining comprises typing 8 HLA loci. In another embodiment, the step of ascertaining comprises typing 9 HLA loci. The HLA assignment of the diseased cells in the human patient that express the one or more antigens of the pathogen or cancer, the human patient, or the donor of a cellular transplant (as the case may be) can be ascertained as described in Section 5.1.1.3, infra.

In addition, to be suitable for therapeutic administration to a human patient in adoptive immunotherapy, the selected T cell line preferably lacks substantial alloreactivity (determined as described in Section 5.1.1.2, infra). In a specific embodiment, each T cell line in the collection of T cell lines lacks substantial alloreactivity (determined as described in Section 5.1.1.2, infra). In another specific embodiment, each T cell line identified in step (a) of the method of selecting a T cell line as described herein lacks substantial alloreactivity (determined as described in Section 5.1.1.2, infra). In a specific embodiment, the identifying step (a) of the method of selecting a T cell line as described herein is identifying those T cell lines in the collection that exhibit a T cell response against one or more antigens of the pathogen or cancer, that lack substantial alloreactivity, and that are restricted by one or more HLA alleles shared with the human patient. In a specific embodiment, the excluding step (b) of the method of selecting a T cell line as described herein further comprises excluding from the T cell lines identified in step (a) those T cell lines that exhibit substantial alloreactivity. In a specific embodiment, the selecting step (c) of the method of selecting a T cell line as described herein further comprises a step (d) of identifying those identified T cell lines remaining after step (b) that lack substantial alloreactivity and the step (c) is selecting for therapeutic administration to said human patient a T cell line from among those identified T cell lines remaining after step (d).

In certain embodiments, the selected T cell line also shares at least 2 HLA alleles (e.g., at least 2 out of 10 HLA alleles) with the human patient (when the human patient has not been the recipient of any cellular transplant) or the Entity (when the human patient has been the recipient of a cellular transplant). The HLA assignment of the diseased cells in the human patient that express the one or more antigens of the pathogen or cancer, the human patient, or the donor of a cellular transplant (as the case may be) and the HLA assignment of a T cell line (either the selected T cell line or a non-selected T cell line in the collection of T cell lines) can be ascertained as described in Section 5.1.1.3, infra. In a specific embodiment, the identifying step (a) of the method of selecting a T cell line as described herein is identifying those T cell lines in the collection that exhibit a T cell response against one or more antigens of the pathogen or cancer, that share at least 2 HLA alleles (e.g., at least 2 out of 10 HLA alleles) with the human patient (when the human patient has not been the recipient of any cellular transplant) or the Entity (when the human patient has been the recipient of a cellular transplant), and that are restricted by one or more HLA alleles shared with the human patient. In a specific embodiment, the excluding step (b) of the method of selecting a T cell line as described herein further comprises excluding from the T cell lines identified in step (a) those T cell lines that do not share at least 2 HLA alleles (e.g., at least 2 out of 10 HLA alleles) with the human patient (when the human patient has not been the recipient of any cellular transplant) or the Entity (when the human patient has been the recipient of a cellular transplant). In a specific embodiment, the selecting step (c) of the method of selecting a T cell line as described herein further comprises a step (d) of identifying those identified T cell lines remaining after step (b) that share at least 2 HLA alleles (e.g., at least 2 out of 10 HLA alleles) with the human patient (when the human patient has not been the recipient of any cellular transplant) or the Entity (when the human patient has been the recipient of a cellular transplant), and the step (c) is selecting for therapeutic administration to said human patient a T cell line from among those identified T cell lines remaining after step (d).

Furthermore, the selected T cell line shall be microbial sterile to be suitable for therapeutic administration. Microbial sterility can be verified by any method known in the art for assaying microbial sterility, for example, by demonstrating negative cultures for bacteria, fungi and mycoplasma and endotoxin levels 5 EU/ml cell dose.

Thus, information as to T cell response (for example, antigen reactivity, such as cytotoxicity, or in vivo clinical efficacy) and HLA restriction, and optionally information as to alloreactivity, HLA type assignment, and/or microbial sterility has been ascertained for each T cell line in the collection of T cell lines (by methods known in the art, for example, methods as described in Koehne et al., 2002, Blood 99:1730-1740; Koehne et al., 2000, Blood 96:109-117; Trivedi et al., 2005, Blood 105:2793-2801; Haque et al., 2007, Blood 110:1123-1131; Hasan et al., 2009, J Immunol 183: 2837-2850; Feuchtinger et al., 2010, Blood 116:4360-4367; Doubrovina et al., 2012, Blood 120:1633-1646; Leen et al., 2013, Blood 121:5113-5123; Papadopoulou et al., 2014, Sci Transl Med 6:242ra83; Sukdolak et al., 2013, Biol Blood Marrow Transplant 19:1480-1492; Koehne et al., 2015, Biol Blood Marrow Transplant 21: 1663-1678; Weren et al., J Immunol Methods, 289:17-26; Shafer-Weaver et al., 2003, J Transl Med 1:14; Nagorsen and Marincola, ed., 2005, Analyzing T Cell Responses: How to Analyze Cellular Immune Responses against Tumor Associated Antigens, Springer Netherlands; International Patent Application Publication No. WO 2016/073550; International Patent Application Publication No. WO 2016/183153; International Patent Application Publication No. WO 2016/209816; International Patent Application Publication No. WO 2017/044678; and/or Sections 5.1.1.1-5.1.1.3, infra), and linked to the identifier of the corresponding T cell line, so as to facilitate the selection of a suitable T cell line from the collection for therapeutic administration to a human patient.

Preferably, the selected T cell line is enriched for T cells. In a specific embodiment, the selected T cell line contains at least 70% T cells. In another specific embodiment, the selected T cell line contains at least 80% T cells. In another specific embodiment, the selected T cell line contains at least 90% T cells. In another specific embodiment, the selected T cell line contains at least 95% T cells. In another specific embodiment, the selected T cell line contains at least 99% T cells. In another specific embodiment, the selected T cell line contains 100% T cells. In a specific embodiment, the selected T cell line contains at least 70% CD3⁺ cells. In another specific embodiment, the selected T cell line contains at least 80% CD3⁺ cells. In another specific embodiment, the selected T cell line contains at least 90% CD3⁺ cells. In another specific embodiment, the selected T cell line contains at least 95% CD3⁺ cells. In another specific embodiment, the selected T cell line contains at least 99% CD3⁺ cells. In another specific embodiment, the selected T cell line contains 100% CD3⁺ cells.

In a specific embodiment, the selected T cell line contains less than 5% natural killer (NK) cells. In another specific embodiment, the selected T cell line contains less than 2% NK cells. In another specific embodiment, the selected T cell line contains less than 1% NK cells. In another specific embodiment, the selected T cell line contains no NK cells.

In a specific embodiment, the selected T cell line contains less than 5% B cells. In another specific embodiment, the selected T cell line contains less than 2% B cells. In another specific embodiment, the selected T cell line contains less than 1% B cells. In another specific embodiment, the selected T cell line contains no B cells.

In a specific embodiment, the selected T cell line comprises CD4⁺ T cells. In another specific embodiment, the selected T cell line comprises CD8⁺ T cells. In another specific embodiment, the selected T cell line comprises both CD8⁺ and CD4⁺ T cells.

In specific embodiments, each T cell line in the collection of T cell lines is enriched for T cells. In a specific embodiment, each T cell line in the collection of T cell lines contains at least 70% T cells. In another specific embodiment, each T cell line in the collection of T cell lines contains at least 80% T cells. In another specific embodiment, each T cell line in the collection of T cell lines contains at least 90% T cells. In another specific embodiment, each T cell line in the collection of T cell lines contains at least 95% T cells. In another specific embodiment, each T cell line in the collection of T cell lines contains at least 99% T cells. In another specific embodiment, each T cell line in the collection of T cell lines contains 100% T cells. In a specific embodiment, each T cell line in the collection of T cell lines contains at least 70% CD3⁺ cells. In another specific embodiment, each T cell line in the collection of T cell lines contains at least 80% CD3⁺ cells. In another specific embodiment, each T cell line in the collection of T cell lines contains at least 90% CD3⁺ cells. In another specific embodiment, each T cell line in the collection of T cell lines contains at least 95% CD3⁺ cells. In another specific embodiment, each T cell line in the collection of T cell lines contains at least 99% CD3⁺ cells. In another specific embodiment, each T cell line in the collection of T cell lines contains 100% CD3⁺ cells.

In a specific embodiment, each T cell line in the collection of T cell lines contains less than 5% natural killer (NK) cells. In another specific embodiment, each T cell line in the collection of T cell lines contains less than 2% NK cells. In another specific embodiment, each T cell line in the collection of T cell lines contains less than 1% NK cells. In another specific embodiment, each T cell line in the collection of T cell lines contains no NK cells.

In a specific embodiment, each T cell line in the collection of T cell lines contains less than 5% B cells. In another specific embodiment, each T cell line in the collection of T cell lines contains less than 2% B cells. In another specific embodiment, each T cell line in the collection of T cell lines contains less than 1% B cells. In another specific embodiment, each T cell line in the collection of T cell lines contains no B cells.

In a specific embodiment, each T cell line in the collection of T cell lines comprises CD4⁺ T cells. In another specific embodiment, each T cell line in the collection of T cell lines comprises CD8⁺ T cells. In another specific embodiment, each T cell line in the collection of T cell lines comprises both CD8⁺ and CD4⁺ T cells.

5.1.1.1. Cytotoxicity and Other Measures of Antigen Reactivity

The antigen reactivity (for example, cytotoxicity) of a T cell line described herein toward fully or partially HLA-matched (relative to the T cell line) target antigen presenting cells can be determined by any assay known in the art to measure T cell mediated antigen reactivity (for example, cytotoxicity), such as, but is not limited to, a method described in Nagorsen and Marincola, ed., 2005, Analyzing T Cell Responses: How to Analyze Cellular Immune Responses against Tumor Associated Antigens, Springer Netherlands. The assay can be performed using the T cell line directly, an aliquot thereof, or a precursor cell population that indicates the antigen reactivity (for example, cytotoxicity) of the T cell line. In a specific embodiment, the antigen reactivity (for example, cytotoxicity) is determined by a standard ⁵¹Cr release assay, an IFN-γ-production assay, a limiting dilution assay to measure CTL precursors (CTLps), a perforin release assay, a granzyme B release assay, or a CD107 mobilization assay, as described in Trivedi et al., 2005, Blood 105:2793-2801; Hasan et al., 2009, J Immunol 183: 2837-2850; Doubrovina et al., 2012, Blood 119:2644-2656; Koehne et al., 2002, Blood 99:1730-1740; Koehne et al., 2000, Blood 96:109-117; Weren et al., J Immunol Methods, 289:17-26; Shafer-Weaver et al., 2003, J Transl Med 1:14; or Nagorsen and Marincola, ed., 2005, Analyzing T Cell Responses: How to Analyze Cellular Immune Responses against Tumor Associated Antigens, Springer Netherlands.

In certain embodiments, a T cell line exhibits a T cell response against one or more antigens of the pathogen or cancer by exhibiting substantial antigen reactivity (for example, cytotoxicity) in vitro toward (e.g., exhibits substantial lysis of) fully or partially HLA-matched (preferably, fully or partially HLA-matched at high resolution) target antigen presenting cells that present the one or more antigens of the pathogen or cancer (e.g., target antigen presenting cells that are loaded with or genetically engineered to express one or more peptides or proteins derived from the one or more antigens of the pathogen or cancer). Preferably, the fully or partially HLA-matched target antigen presenting cells are fully HLA-matched target antigen presenting cells (e.g., target antigen presenting cells derived from the human donor of the population of human blood cells used to generate the T cell line). In specific embodiments, the T cell line exhibits lysis of greater than or equal to 20%, 25%, 30%, 35%, or 40% of the fully or partially HLA-matched target antigen presenting cells that present the one or more antigens of the pathogen or cancer (e.g., target antigen presenting cells that are loaded with or genetically engineered to express one or more peptides or proteins derived from the one or more antigens of the pathogen or cancer). In a specific embodiment, the T cell line exhibits lysis of greater than or equal to 20% of the fully or partially HLA-matched target antigen presenting cells that present the one or more antigens of the pathogen or cancer (e.g., target antigen presenting cells that are loaded with or genetically engineered to express one or more peptides or proteins derived from the one or more antigens of the pathogen or cancer). In another specific embodiment, the T cell line exhibits lysis of greater than or equal to 25% of the fully or partially HLA-matched target antigen presenting cells that present the one or more antigens of the pathogen or cancer (e.g., target antigen presenting cells that are loaded with or genetically engineered to express one or more peptides or proteins derived from the one or more antigens of the pathogen or cancer).

In a specific embodiment, a T cell line exhibits a T cell response when the antigen reactivity (for example, cytotoxicity) exhibited by the T cell line is at least 2-fold higher than the antigen reactivity (for example, cytotoxicity) normally exhibited by unselected donor lymphocytes from human blood, e.g., as used in donor lymphocyte infusions. In another specific embodiment, a T cell line exhibits a T cell response when the antigen reactivity (for example, cytotoxicity) exhibited by the T cell line is at least 5-fold higher than the antigen reactivity (for example, cytotoxicity) normally exhibited by unselected donor lymphocytes used in donor lymphocyte infusions. In another specific embodiment, a T cell line exhibits a T cell response when the antigen reactivity (for example, cytotoxicity) exhibited by the T cell line is at least 10-fold higher than the antigen reactivity (for example, cytotoxicity) normally exhibited by unselected donor lymphocytes used in donor lymphocyte infusions. In another specific embodiment, a T cell line exhibits a T cell response when the antigen reactivity (for example, cytotoxicity) exhibited by the T cell line is at least 20-fold higher than the antigen reactivity (for example, cytotoxicity) normally exhibited by unselected donor lymphocytes used in donor lymphocyte infusions. In another specific embodiment, a T cell line exhibits a T cell response when the antigen reactivity (for example, cytotoxicity) exhibited by the T cell line is at least 50-fold higher than the antigen reactivity (for example, cytotoxicity) normally exhibited by unselected donor lymphocytes used in donor lymphocyte infusions. In another specific embodiment, a T cell line exhibits a T cell response when the antigen reactivity (for example, cytotoxicity) exhibited by the T cell line is at least 100-fold higher than the antigen reactivity (for example, cytotoxicity) normally exhibited by unselected donor lymphocytes used in donor lymphocyte infusions. In another specific embodiment, a T cell line exhibits a T cell response when the antigen reactivity (for example, cytotoxicity) exhibited by the T cell line is at least 200-fold higher than the antigen reactivity (for example, cytotoxicity) normally exhibited by unselected donor lymphocytes used in donor lymphocyte infusions. In another specific embodiment, a T cell line exhibits a T cell response when the antigen reactivity (for example, cytotoxicity) exhibited by the T cell line is at least 500-fold higher than the antigen reactivity (for example, cytotoxicity) normally exhibited by unselected donor lymphocytes used in donor lymphocyte infusions. In another specific embodiment, a T cell line exhibits a T cell response when the antigen reactivity (for example, cytotoxicity) exhibited by the T cell line is at least 1000-fold higher than the antigen reactivity (for example, cytotoxicity) normally exhibited by unselected donor lymphocytes used in donor lymphocyte infusions.

Target antigen presenting cells that can be used in the antigen reactivity (for example, cytotoxicity) assay include, but are not limited to, dendritic cells, phytohemagglutinin (PHA)-lymphoblasts, macrophages, B-cells that generate antibodies, EBV-BLCL cells, and artificial antigen presenting cells (AAPCs). Target antigen presenting cells that can be used in the antigen reactivity (for example, cytotoxicity) assay can be either professional antigen presenting cells or non-professional antigen presenting cells.

In a specific embodiment, multiple iterations of an antigen reactivity (for example, cytotoxicity) assay are performed, wherein different populations of target antigen presenting cells are used that present the same target antigen(s) of the pathogen or cancer in the multiple iterations of the assay, and the antigen reactivity of the T cell line preferably is the average value of the different iterations of the assay. The multiple iterations of an antigen reactivity (for example, cytotoxicity) assay preferably are performed under essentially the same conditions. The different populations of target antigen presenting cells can be of different types (for example, one population of target antigen presenting cells can be PHA-lymphoblasts, while another population of target antigen presenting cells can be EBV-BLCL cells), but preferably are of the same type (for example, all of the different populations of target antigen presenting cells are PHA-lymphoblasts).

In specific embodiments, the fully or partially HLA-matched target antigen presenting cells used in the antigen reactivity (for example, cytotoxicity) assay are loaded with a pool of peptides derived from the one or more antigens of the pathogen or cancer. The pool of peptides, can be, for example, a pool of overlapping peptides (e.g., pentadecapeptides) spanning the sequence(s) of the one or more antigens of the pathogen or cancer.

5.1.1.2. Alloreactivity

Alloreactivity of a T cell line described herein can be measured using an antigen reactivity (for example, cytotoxicity) assay known in the art to measure T cell mediated antigen reactivity (for example, cytotoxicity), such as, but is limited to, a standard ⁵¹Cr release assay, an IFN-γ-production assay, a limiting dilution assay to measure CTL precursors (CTLps), a perforin release assay, a granzyme B release assay, a CD107 mobilization assay, or any other antigen reactivity assay as described in Section 5.1.1.1, but with target antigen presenting cells that do not present the one or more antigens of the pathogen or cancer (e.g., target antigen presenting cells that are not loaded with or genetically engineered to express one or more peptides or proteins derived from the one or more antigens of the pathogen or cancer), and/or completely HLA-mismatched (preferably, completely HLA-mismatched at low resolution and/or completely HLA supertype mismatched, wherein the HLA supertypes are classified on the basis of their main anchor specificity (see, for example, as described in Sidney et al., 2008, BMC Immunology 9:1)) (relative to the T cell line) target antigen presenting cells. The assay can be performed using the T cell line directly, an aliquot thereof, or a precursor cell population that indicates the alloreactivity of the T cell line. A T cell line that lacks substantial alloreactivity results generally in the absence of graft-versus-host disease (GvHD) when administered to a human patient.

In certain embodiments, a T cell line lacks substantial alloreactivity as determined by lacking substantial antigen reactivity (for example, cytotoxicity) in vitro toward target antigen presenting cells that do not present the one or more antigens of the pathogen or cancer (e.g., target antigen presenting cells that are not loaded with or genetically engineered to express one or more peptides or proteins derived from the one or more antigens of the pathogen or cancer). In a specific embodiment, such target antigen presenting cells are completely HLA-mismatched (preferably, completely HLA-mismatched at low resolution and/or completely HLA supertype mismatched, wherein the HLA supertypes are classified on the basis of their main anchor specificity (see, for example, as described in Sidney et al., 2008, BMC Immunology 9:1)) relative to the T cell line. In another specific embodiment, such target antigen presenting cells are fully or partially HLA-matched relative to the T cell line (e.g., target antigen presenting cells derived from the human donor of the population of human blood cells used to generate the population of human cells). In specific embodiments, the T cell line lyses less than or equal to 15%, 10%, 5%, 2%, or 1% of target antigen presenting cells that do not present the one or more antigens of the pathogen or cancer (e.g., target antigen presenting cells that are not loaded with or genetically engineered to express one or more peptides or proteins derived from the one or more antigens of the pathogen or cancer). In a specific embodiment, the T cell line lyses less than or equal to 10% of target antigen presenting cells that do not present the one or more antigens of the pathogen or cancer (e.g., target antigen presenting cells that are not loaded with or genetically engineered to express one or more peptides or proteins derived from the one or more antigens of the pathogen or cancer). In another specific embodiment, the T cell line lyses less than or equal to 10% of target antigen presenting cells that do not present the one or more antigens of the pathogen or cancer (e.g., target antigen presenting cells that are not loaded with or genetically engineered to express one or more peptides or proteins derived from the one or more antigens of the pathogen or cancer). In another specific embodiment, the T cell line lyses less than or equal to 5% of target antigen presenting cells that do not present the one or more antigens of the pathogen or cancer (e.g., target antigen presenting cells that are not loaded with or genetically engineered to express one or more peptides or proteins derived from the one or more antigens of the pathogen or cancer).

In certain embodiments, a T cell line lacks substantial alloreactivity as determined by lacking substantial antigen reactivity (for example, cytotoxicity) in vitro toward completely HLA-mismatched (preferably, completely HLA-mismatched at low resolution and/or completely HLA supertype mismatched, wherein the HLA supertypes are classified on the basis of their main anchor specificity (see, for example, as described in Sidney et al., 2008, BMC Immunology 9:1)) (relative to the T cell line) target antigen presenting cells. In a specific embodiment, such target antigen presenting cells present the one or more antigens of the pathogen or cancer (e.g., are loaded with or genetically engineered to express one or more peptides or proteins derived from the one or more antigens of the pathogen or cancer). In a preferred embodiment, such target antigen presenting cells do not present the one or more antigens of the pathogen or cancer (e.g., are not loaded with or genetically engineered to express one or more peptides or proteins derived from the one or more antigens of the pathogen or cancer). In specific embodiments, the T cell line lyses less than or equal to 15%, 10%, 5%, 2%, or 1% of completely HLA-mismatched (relative to the T cell line) target antigen presenting cells. In a specific embodiment, the T cell line lyses less than or equal to 10% of completely HLA-mismatched (relative to the T cell line) target antigen presenting cells. In another specific embodiment, the T cell line lyses less than or equal to 5% of completely HLA-mismatched (relative to the T cell line) target antigen presenting cells.

In certain embodiments, a T cell line lacks substantial alloreactivity as determined by lacking substantial antigen reactivity (for example, cytotoxicity) in vitro toward target antigen presenting cells that do not present the one or more antigens of the pathogen or cancer (e.g., target antigen presenting cells that are not loaded with or genetically engineered to express one or more peptides or proteins derived from the one or more antigens of the pathogen or cancer), as described above, and lacking substantial antigen reactivity (for example, cytotoxicity) in vitro toward completely HLA-mismatched (preferably, completely HLA-mismatched at low resolution and/or completely HLA supertype mismatched, wherein the HLA supertypes are classified on the basis of their main anchor specificity (see, for example, as described in Sidney et al., 2008, BMC Immunology 9:1)) target antigen presenting cells as described above.

In a specific embodiment, a T cell line lacks substantial alloreactivity when the alloreactivity exhibited by the T cell line is at least 2-fold lower than the alloreactivity normally exhibited by unselected donor lymphocytes used in donor lymphocyte infusions. In another specific embodiment, a T cell line lacks substantial alloreactivity when the alloreactivity exhibited by the T cell line is at least 5-fold lower than the alloreactivity normally exhibited by unselected donor lymphocytes used in donor lymphocyte infusions. In another specific embodiment, a T cell line lacks substantial alloreactivity when the alloreactivity exhibited by the T cell line is at least 10-fold lower than the alloreactivity normally exhibited by unselected donor lymphocytes used in donor lymphocyte infusions. In another specific embodiment, a T cell line lacks substantial alloreactivity when the alloreactivity exhibited by the T cell line is at least 20-fold lower than the alloreactivity normally exhibited by unselected donor lymphocytes used in donor lymphocyte infusions. In another specific embodiment, a T cell line lacks substantial alloreactivity when the alloreactivity exhibited by the T cell line is at least 50-fold lower than the alloreactivity normally exhibited by unselected donor lymphocytes used in donor lymphocyte infusions. In another specific embodiment, a T cell line lacks substantial alloreactivity when the alloreactivity exhibited by the T cell line is at least 100-fold lower than the alloreactivity normally exhibited by unselected donor lymphocytes used in donor lymphocyte infusions. In another specific embodiment, a T cell line lacks substantial alloreactivity when the alloreactivity exhibited by the T cell line is at least 200-fold lower than the alloreactivity normally exhibited by unselected donor lymphocytes used in donor lymphocyte infusions. In another specific embodiment, a T cell line lacks substantial alloreactivity when the alloreactivity exhibited by the T cell line is at least 500-fold lower than the alloreactivity normally exhibited by unselected donor lymphocytes used in donor lymphocyte infusions. In another specific embodiment, a T cell line lacks substantial alloreactivity when the alloreactivity exhibited by the T cell line is at least 1000-fold lower than the alloreactivity normally exhibited by unselected donor lymphocytes used in donor lymphocyte infusions.

In a specific embodiment, a T cell line lacks substantial alloreactivity when the T cell line described herein contains less than 500, less than 300, or less than 100 alloreactive cytotoxic T lymphocyte precursors (CTLps) per million cells, when the amount of alloreactive CTLps per million cells is determined to be the average amount of alloreactive CTLps per million cells determined in N limiting dilution assays, each assay using a different population of target antigen presenting cells that are completely HLA-mismatched (preferably, completely HLA-mismatched at low resolution and/or completely HLA supertype mismatched, wherein the HLA supertypes are classified on the basis of their main anchor specificity (see, for example, as described in Sidney et al., 2008, BMC Immunology 9:1)) relative to the T cell line, wherein each different population of target antigen presenting cells is of different HLA type, wherein N is an integer greater than 1. In another specific embodiment, a T cell line lacks substantial alloreactivity when the T cell line described herein contains less than 100 alloreactive cytotoxic T lymphocyte precursors (CTLps) per million cells, when the amount of alloreactive CTLps per million cells is determined to be the average amount of alloreactive CTLps per million cells determined in N limiting dilution assays, each assay using a different population of target antigen presenting cells that are completely HLA-mismatched (preferably, completely HLA-mismatched at low resolution and/or completely HLA supertype mismatched, wherein the HLA supertypes are classified on the basis of their main anchor specificity (see, for example, as described in Sidney et al., 2008, BMC Immunology 9:1)) relative to the T cell line, wherein each different population of target antigen presenting cells is of different HLA type, wherein N is an integer greater than 1. In another specific embodiment, a T cell line lacks substantial alloreactivity when the T cell line described herein contains less than 150 alloreactive cytotoxic T lymphocyte precursors (CTLps) per million cells, when the amount of alloreactive CTLps per million cells is determined to be the average amount of alloreactive CTLps per million cells determined in N limiting dilution assays, each assay using a different population of target antigen presenting cells that are completely HLA-mismatched (preferably, completely HLA-mismatched at low resolution and/or completely HLA supertype mismatched, wherein the HLA supertypes are classified on the basis of their main anchor specificity (see, for example, as described in Sidney et al., 2008, BMC Immunology 9:1)) relative to the T cell line, wherein each different population of target antigen presenting cells is of different HLA type, wherein N is an integer greater than 1. In a specific embodiment, N is greater than 2. In another specific embodiment, N is greater than 3. In another specific embodiment, N is greater than 4. In another specific embodiment, N equals 2. In another specific embodiment, N equals 3. In another specific embodiment, N equals 4. Preferably, N equals 5. While each population of target antigen presenting cells used in the limiting dilution assays may present the one or more antigens of the pathogen or cancer (e.g., are loaded with or genetically engineered to express one or more peptides or proteins derived from the one or more antigens of the pathogen or cancer), in a preferred embodiment, each population of target antigen presenting cells used in the limiting dilution assays does not present the one or more antigens of the pathogen or cancer (e.g., are not loaded with or genetically engineered to express one or more peptides or proteins derived from the one or more antigens of the pathogen or cancer). By way of example, when the target antigen presenting cells do not express and are not loaded with one or more peptides or proteins derived from the one or more antigens of the pathogen or cancer, the target antigen presenting cells cannot and thus do not present such antigens. The limiting dilution assays can be performed by any method known in the art, for example, as described in Doubrovina et al., 2012, Blood 119:2644-2656; Koehne et al., 2002, Blood 99:1730-1740; or Koehne et al., 2000, Blood 96:109-117. Preferably, the limiting dilution assays are performed under essentially the same conditions.

Target antigen presenting cells that can be used in the alloreactivity assay include, but are not limited to, dendritic cells, phytohemagglutinin (PHA)-lymphoblasts, macrophages, B-cells that generate antibodies, EBV-BLCL cells, and artificial antigen presenting cells (AAPCs). If the T cell line is generated by ex vivo sensitizing human T cells to one or more antigens of the pathogen or cancer presented by EBV-BLCL cells, in a preferred embodiment, the target antigen presenting cells used in the alloreactivity assay are not EBV-BLCL cells. In a specific aspect of such preferred embodiment, the target antigen presenting cells used in the alloreactivity assay are dendritic cells or PHA-lymphoblasts. Target antigen presenting cells that can be used in the alloreactivity assay can be either professional antigen presenting cells or non-professional antigen presenting cells.

In a specific embodiment, multiple iterations of an alloreactivity assay are performed, wherein different populations of target antigen presenting cells are used in the multiple iterations of the assay, the alloreactivity of the T cell line preferably is the average value of the different iterations of the assay. The multiple iterations of an alloreactivity assay preferably are performed under essentially the same conditions. The different populations of target antigen presenting cells can be of different types (for example, one population of target antigen presenting cells can be PHA-lymphoblasts, while another population of target antigen presenting cells can be EBV-BLCL cells), but preferably are of the same type (for example, all of the different populations of target antigen presenting cells are PHA-lymphoblasts).

5.1.1.3. HLA Type and Restriction

According to the invention, the T cell line selected according to a method described herein is restricted to more than one HLA alleles shared with the human patient (when the human patient has not been the recipient of any cellular transplant) or the Entity (when the human patient has been the recipient of a cellular transplant). In specific embodiments, the T cell line selected also shares at least 2 HLA alleles with the human patient (when the human patient has not been the recipient of any cellular transplant) or the Entity (when the human patient has been the recipient of a cellular transplant) (for example, at least 2 out of 8 HLA alleles (such as two HLA-A alleles, two HLA-B alleles, two HLA-C alleles, and two HLA-DR alleles (preferably two HLA-DRB1 alleles), or preferably at least 2 out of 10 HLA alleles (such as two HLA-A alleles, two HLA-B alleles, two HLA-C alleles, two HLA-DR alleles (preferably two HLA-DRB1 alleles), and two HLA-DQ alleles (preferably two HLA-DQB1 alleles)).

The HLA allele(s) by which a T cell line (either the selected T cell line or a non-selected T cell line in the collection of T cell lines) described herein is restricted can be determined by any method known in the art, for example, as described in Trivedi et al., 2005, Blood 105:2793-2801; Barker et al., 2010, Blood 116:5045-5049; Hasan et al., 2009, J Immunol, 183:2837-2850; Doubrovina et al., 2012, Blood 120:1633-1646; International Patent Application Publication No. WO 2016/073550; International Patent Application Publication No. WO 2016/183153; International Patent Application Publication No. WO 2016/209816; or International Patent Application Publication No. WO 2017/044678. The determination can be performed using the T cell line directly, an aliquot thereof, or a precursor cell population that indicates the HLA allele(s) by which the T cell line is restricted.

The HLA assignment (i.e., the HLA loci type) of the diseased cells in the human patient that express the one or more antigens of the pathogen or cancer, the human patient, or the donor of a cellular transplant (as the case may be) and the HLA assignment of a T cell line described herein (either the selected T cell line or a non-selected T cell line in the collection of T cell lines) can be ascertained (i.e., typed) by any method known in the art for typing HLA alleles. Non-limiting exemplary methods for ascertaining the HLA assignment can be found in ASHI Laboratory Manual, Edition 4.2 (2003), American Society for Histocompatibility and Immunogenetics; ASHI Laboratory Manual, Supplements 1 (2006) and 2 (2007), American Society for Histocompatibility and Immunogenetics; Hurley, “DNA-based typing of HLA for transplantation.” in Leffell et al., eds., 1997, Handbook of Human Immunology, Boca Raton: CRC Press; Dunn, 2011, Int J Immunogenet 38:463-473; Erlich, 2012, Tissue Antigens, 80:1-11; Bontadini, 2012, Methods, 56:471-476; and Lange et al., 2014, BMC Genomics 15: 63. In specific embodiments, at least 4 HLA loci (preferably HLA-A, HLA-B, HLA-C, and HLA-DR (preferably HLA-DRB1)) are typed. In a specific embodiment, 4 HLA loci (preferably HLA-A, HLA-B, HLA-C, and HLA-DR (preferably HLA-DRB1)) are typed. In another specific embodiment, 5 HLA loci (preferably HLA-A, HLA-B, HLA-C, HLA-DR (preferably HLA-DRB1), and HLA-DQ (preferably HLA-DQB1)) are typed. In another specific embodiment, 6 HLA loci are typed. In another specific embodiment, 7 HLA loci are typed. In another specific embodiment, 8 HLA loci are typed. In another specific embodiment, 9 HLA loci are typed.

In general, high-resolution typing is preferable for HLA typing. The high-resolution typing can be performed by any method known in the art, for example, as described in ASHI Laboratory Manual, Edition 4.2 (2003), American Society for Histocompatibility and Immunogenetics; ASHI Laboratory Manual, Supplements 1 (2006) and 2 (2007), American Society for Histocompatibility and Immunogenetics; Flomenberg et al., Blood, 104:1923-1930; Kogler et al., 2005, Bone Marrow Transplant, 36:1033-1041; Lee et al., 2007, Blood 110:4576-4583; Erlich, 2012, Tissue Antigens, 80:1-11; Lank et al., 2012, BMC Genomics 13:378; or Gabriel et al., 2014, Tissue Antigens, 83:65-75.

The HLA assignment of a T cell line (either the selected T cell line or a non-selected T cell line in the collection of T cell lines) can be performed using the T cell line directly, an aliquot thereof, or a precursor cell population that indicates the HLA assignment of the T cell line.

The HLA assignment of the diseased cells in the human patient that express the one or more antigens of the pathogen or cancer can be performed using a sample of the diseased cells.

The HLA assignment of the human patient or the donor of a cellular transplant (as the case may be) can be performed using a tissue or cell sample from the individual.

As described above, when the human patient has not been the recipient of any cellular transplant, the method of selecting a T cell line described herein comprises: (a) identifying those T cell lines in the collection that exhibit a T cell response against one or more antigens of the pathogen or cancer and that are restricted by one or more HLA alleles shared with the human patient; (b) excluding from the T cell lines identified in step (a) those T cell lines that exhibit a T cell response against said one or more antigens of the pathogen or cancer and that are restricted by only one HLA allele shared with the human patient; and (c) selecting for therapeutic administration to said human patient a T cell line from among those identified T cell lines remaining after step (b).

As described above, when the human patient has been the recipient of a cellular transplant, the method of selecting a T cell line described herein comprises: (a) identifying those T cell lines in the collection that exhibit a T cell response against one or more antigens of the pathogen or cancer and that are restricted by one or more HLA alleles shared with an entity selected from the group consisting of (i) the diseased cells in the human patient that express the one or more antigens of the pathogen or cancer, (ii) the human patient, (iii) the donor of the cellular transplant, and (iv) both the human patient and the donor of the cellular transplant); (b) excluding from the T cell lines identified in step (a) those T cell lines that exhibit a T cell response against said one or more antigens of the pathogen or cancer and that are restricted by only one HLA allele shared with the entity; and (c) selecting for therapeutic administration to said human patient a T cell line from among those identified T cell lines remaining after step (b). As noted hereinabove, the entity that is selected from the group consisting of (i) the diseased cells in the human patient that express the one or more antigens of the pathogen or cancer, (ii) the human patient, (iii) the donor of the cellular transplant, and (iv) both the human patient and the donor of the cellular transplant; in such a method shall be referred to herein as the “Entity,” for purposes of convenience. Guidance that can be used for the selection of the Entity is provided below.

When the HLA assignment of the diseased cells in the human patient that express the one or more antigens of the pathogen or cancer is known, the preferred choice of Entity is the diseased cells.

When the HLA assignment of the diseased cells is unknown (for example, when HLA typing of a sample of the diseased cells has not been done or is not feasible), but the origin of the diseased cells in the human patient that express the one or more antigens of the pathogen or cancer is known to be the human patient (as opposed to the donor of the cellular transplant), the preferred choice of Entity is the human patient.

When the HLA assignment of the diseased cells is unknown (for example, when HLA typing of a sample of the diseased cells has not been done or is not feasible), but the origin of the diseased cells in the human patient that express the one or more antigens of the pathogen or cancer is known to be the donor of the cellular transplant, the preferred choice of Entity is the donor of the cellular transplant.

When the HLA assignment of the diseased cells is unknown (for example, when HLA typing of a sample of the diseased cells has not been done or is not feasible), but the origin of the diseased cells in the human patient that express the one or more antigens of the pathogen or cancer is known to be both the human patient and the donor of the cellular transplant (i.e., part of the diseased cells originated from the human patient while another part of the diseased cells originated from the donor of the cellular transplant), the preferred choice of Entity is both the human patient and the donor of the cellular transplant.

The origin of the diseased cells in the human patient can be determined by any method known in the art, for example, by analyzing variable tandem repeats (VTRs) (which is a method that uses unique DNA signature of small DNA sequences of different people to distinguish between the recipient and the donor of a cellular transplant), or by looking for the presence or absence of chromosome Y if the donor and the recipient of a cellular transplant are of different sexes (which is done by cytogenetics or by FISH (fluorescence in situ hybridization)).

When the HLA assignment of the diseased cells is unknown (for example, when HLA typing of a sample of the diseased cells has not been done or is not feasible), and the origin of the diseased cells in the human patient that express the one or more antigens of the pathogen or cancer is more likely than not to be originated from the human patient, preferably the Entity is the human patient. One example of such a case can be where the disease or disorder or the cancer to be treated is EBV-associated post-transplant lymphoproliferative disorder (PTLD) (EBV-PTLD) post SOT (see, for example, Kinch et al., 2014, American Journal of Transplantation 14:2838-2845).

When the HLA assignment of the diseased cells is unknown (for example, when HLA typing of a sample of the diseased cells has not been done or is not feasible), and the origin of the diseased cells in the human patient that express the one or more antigens of the pathogen or cancer is more likely than not to be originated from the donor of the cellular transplant, preferably the Entity is the donor of the cellular transplant. One example of such a case can be where the disease or disorder or the cancer to be treated is EBV-PTLD post HSCT (see, for example, Kinch et al., 2014, American Journal of Transplantation 14:2838-2845).

When the HLA assignment of the diseased cells is unknown (for example, when HLA typing of a sample of the diseased cells has not been done or is not feasible), and the origin of the diseased cells in the human patient that express the one or more antigens of the pathogen or cancer is more likely than not to be originated from both the human patient and the donor of the cellular transplant, preferably the Entity is both the human patient and the donor of the cellular transplant.

When the HLA assignment of the diseased cells is unknown (for example, when HLA typing of a sample of the diseased cells has not been done or is not feasible), and the origin of the diseased cells in the human patient that express the one or more antigens of the pathogen or cancer is unknown (for example, when determining the origin of the diseased cells has not been done or is not feasible), and it is unknown whether the diseased cells that express the one or more antigens of the pathogen or cancer are more likely than not to be originated from the human patient, the donor of the cellular transplant, or both the human patient and the donor of the cellular transplant, preferably the Entity is both the human patient and the donor of the cellular transplant. However, if no T cell line can be selected that meets the selection criteria described herein when Entity is both the human patient and the donor of the cellular transplant, the the human patient or the donor of the cellular transplant can be used as the Entity.

While guidance for selection of the Entity is provided above, such selection is within the discretion of the treating physician according to his/her judgment.

5.2. Therapeutic Uses of Selected T Cell Lines

In another aspect, provided herein is a method of treating a disease or disorder associated with a pathogen or treating a cancer in a human patient, comprising: (a) selecting a T cell line for therapeutic administration to the human patient according to a method of selecting a T cell line as described in Section 5.1; and (b) administering to the human patient a population of human cells comprising antigen-specific T cells that are specific for one or more antigens of the pathogen or cancer, which population of human cells is derived from the selected T cell line.

5.2.1. Administration and Dosage of the Population of Human Cells

The route of administration of the population of human cells comprising antigen-specific T cells and the amount to be administered to the human patient can be determined based on the nature of the disease, condition of the human patient and the knowledge of the physician. Generally, the administration of the population of human cells is intravenous. In certain embodiments, the method of treating comprises infusing to the human patient the population of human cells comprising antigen-specific T cells. In specific embodiments, the infusing is by bolus intravenous infusion.

In specific embodiments, the method described herein comprises administering to the human patient the population of human cells comprising antigen-specific T cells, at a dose that is at least 1×10² cells of the population of human cells comprising antigen-specific T cells per kg of the human patient. In specific embodiments, the method described herein comprises administering to the human patient the population of human cells comprising antigen-specific T cells, at a dose that is at least 1×10³ cells of the population of human cells comprising antigen-specific T cells per kg of the human patient. In specific embodiments, the method described herein comprises administering to the human patient the population of human cells comprising antigen-specific T cells, at a dose that is at least 1×10⁴ cells of the population of human cells comprising antigen-specific T cells per kg of the human patient. In specific embodiments, the method described herein comprises administering to the human patient the population of human cells comprising antigen-specific T cells, at a dose that is at least 1×10⁵ cells of the population of human cells comprising antigen-specific T cells per kg of the human patient. In a specific embodiment, the method of treating comprises administering to the human patient the population of human cells comprising antigen-specific T cells, at a dose that is about 1×10² cells of the population of human cells comprising antigen-specific T cells per kg of the human patient. In another specific embodiment, the method of treating comprises administering to the human patient the population of human cells comprising antigen-specific T cells, at a dose that is about 5×10² cells of the population of human cells comprising antigen-specific T cells per kg of the human patient. In another specific embodiment, the method of treating comprises administering to the human patient the population of human cells comprising antigen-specific T cells, at a dose that is about 1×10³ cells of the population of human cells comprising antigen-specific T cells per kg of the human patient. In another specific embodiment, the method of treating comprises administering to the human patient the population of human cells comprising antigen-specific T cells, at a dose that is about 5×10³ cells of the population of human cells comprising antigen-specific T cells per kg of the human patient. In another specific embodiment, the method of treating comprises administering to the human patient the population of human cells comprising antigen-specific T cells, at a dose that is about 1×10⁴ cells of the population of human cells comprising antigen-specific T cells per kg of the human patient. In another specific embodiment, the method of treating comprises administering to the human patient the population of human cells comprising antigen-specific T cells, at a dose that is about 5×10⁴ cells of the population of human cells comprising antigen-specific T cells per kg of the human patient. In another specific embodiment, the method of treating comprises administering to the human patient the population of human cells comprising antigen-specific T cells, at a dose that is about 5×10⁵ cells of the population of human cells comprising antigen-specific T cells per kg of the human patient. In another specific embodiment, the method of treating comprises administering to the human patient the population of human cells comprising antigen-specific T cells, at a dose that is about 1×10⁶ cells of the population of human cells comprising antigen-specific T cells per kg of the human patient. In another specific embodiment, the method of treating comprises administering to the human patient the population of human cells comprising antigen-specific T cells, at a dose that is about 2×10⁶ cells of the population of human cells comprising antigen-specific T cells per kg of the human patient. In another specific embodiment, the method of treating comprises administering to the human patient the population of human cells comprising antigen-specific T cells, at a dose that is about 3×10⁶ cells of the population of human cells comprising antigen-specific T cells per kg of the human patient. In another specific embodiment, the method of treating comprises administering to the human patient the population of human cells comprising antigen-specific T cells, at a dose that is about 4×10⁶ cells of the population of human cells comprising antigen-specific T cells per kg of the human patient. In another specific embodiment, the method of treating comprises administering to the human patient the population of human cells comprising antigen-specific T cells, at a dose that is about 5×10⁶ cells of the population of human cells comprising antigen-specific T cells per kg of the human patient. In another specific embodiment, the method of treating comprises administering to the human patient the population of human cells comprising antigen-specific T cells, at a dose that is about 6×10⁶ cells of the population of human cells comprising antigen-specific T cells per kg of the human patient. In another specific embodiment, the method of treating comprises administering to the human patient the population of human cells comprising antigen-specific T cells, at a dose that is about 1×10⁷ cells of the population of human cells comprising antigen-specific T cells per kg of the human patient. In another specific embodiment, the method of treating comprises administering to the human patient the population of human cells comprising antigen-specific T cells, at a dose that is about 1×10² to 5×10² cells of the population of human cells comprising antigen-specific T cells per kg of the human patient. In another specific embodiment, the method of treating comprises administering to the human patient the population of human cells comprising antigen-specific T cells, at a dose that is about 5×10² to 1×10³ cells of the population of human cells comprising antigen-specific T cells per kg of the human patient. In another specific embodiment, the method of treating comprises administering to the human patient the population of human cells comprising antigen-specific T cells, at a dose that is about 1×10³ to 5×10³ cells of the population of human cells comprising antigen-specific T cells per kg of the human patient. In another specific embodiment, the method of treating comprises administering to the human patient the population of human cells comprising antigen-specific T cells, at a dose that is about 5×10³ to 1×10⁴ cells of the population of human cells comprising antigen-specific T cells per kg of the human patient. In another specific embodiment, the method of treating comprises administering to the human patient the population of human cells comprising antigen-specific T cells, at a dose that is about 1×10⁴ to 5×10⁴ cells of the population of human cells comprising antigen-specific T cells per kg of the human patient. In another specific embodiment, the method of treating comprises administering to the human patient the population of human cells comprising antigen-specific T cells, at a dose that is about 5×10⁴ to 1×10⁵ cells of the population of human cells comprising antigen-specific T cells per kg of the human patient. In another specific embodiment, the method of treating comprises administering to the human patient the population of human cells comprising antigen-specific T cells, at a dose that is about 1×10⁵ to 5×10⁵ cells of the population of human cells comprising antigen-specific T cells per kg of the human patient. In another specific embodiment, the method of treating comprises administering to the human patient the population of human cells comprising antigen-specific T cells, at a dose that is about 5×10⁵ to 1×10⁶ cells of the population of human cells comprising antigen-specific T cells per kg of the human patient. In another specific embodiment, the method of treating comprises administering to the human patient the population of human cells comprising antigen-specific T cells, at a dose that is about 1×10⁶ to 5×10⁶ cells of the population of human cells comprising antigen-specific T cells per kg of the human patient. In another specific embodiment, the method of treating comprises administering to the human patient the population of human cells comprising antigen-specific T cells, at a dose that is about 1×10⁶ to 2×10⁶ cells of the population of human cells comprising antigen-specific T cells per kg of the human patient. In another specific embodiment, the method of treating comprises administering to the human patient the population of human cells comprising antigen-specific T cells, at a dose that is about 2×10⁶ to 5×10⁶ cells of the population of human cells comprising antigen-specific T cells per kg of the human patient. In another specific embodiment, the method of treating comprises administering to the human patient the population of human cells comprising antigen-specific T cells, at a dose that is about 5×10⁶ to 1×10⁷ cells of the population of human cells comprising antigen-specific T cells per kg of the human patient.

In a preferred embodiment, the method of treating comprises administering to the human patient the population of human cells comprising antigen-specific T cells at the dose described above weekly. In a specific embodiment, the method of treating comprises administering to the human patient the population of human cells comprising antigen-specific T cells at the dose described above twice weekly. In another specific embodiment, the method of treating comprises administering to the human patient the population of human cells comprising antigen-specific T cells at the dose described above biweekly. In another specific embodiment, the method of treating comprises administering to the human patient the population of human cells comprising antigen-specific T cells at the dose described above every three weeks.

In certain embodiments, the method of treating comprises administering to the human patient at least 2 doses of the population of human cells comprising antigen-specific T cells. In specific embodiments, the method of treating comprises administering to the human patient 2, 3, 4, 5, or 6 doses of the population of human cells comprising antigen-specific T cells. In a specific embodiment, the method of treating comprises administering to the human patient 2 doses of the population of human cells comprising antigen-specific T cells. In another specific embodiment, the method of treating comprises administering to the human patient 3 doses of the population of human cells comprising antigen-specific T cells. In another specific embodiment, the method of treating comprises administering to the human patient 4 doses of the population of human cells comprising antigen-specific T cells.

In specific embodiments, the method of treating comprises administering to the human patient at least two cycles (e.g., 2, 3, 4, 5, or 6 cycles) of one dose per week of the population of human cells comprising antigen-specific T cells for at least two consecutive weeks (e.g., 2, 3, 4, 5, or 6 consecutive weeks), each cycle separated by a washout period during which no dose of the population of human cells comprising antigen-specific T cells is administered. In a specific embodiment, the at least two consecutive weeks are 2 consecutive weeks. In a preferred embodiment, the at least two consecutive weeks are 3 consecutive weeks. In another specific embodiment, the at least two consecutive weeks are 4 consecutive weeks. In another specific embodiment, the method of treating comprises administering to the human patient two, three, four, five, or six cycles of one dose per week of the population of human cells comprising antigen-specific T cells for three consecutive weeks, each cycle separated by a washout period during which no dose of the population of human cells comprising antigen-specific T cells is administered. In another specific embodiment, the method of treating comprises administering to the human patient a first cycle of one dose per week of the population of human cells comprising antigen-specific T cells for 3 consecutive weeks followed by a washout period during which no dose of the population of human cells comprising antigen-specific T cells is administered, followed by a second cycle of said one dose per week of the population of human cells comprising antigen-specific T cells for 3 consecutive weeks. In specific embodiments, the washout period is at least about 1 week (e.g., about 1-6 weeks). In specific embodiments, the washout period is about 1, 2, 3, or 4 weeks. In specific embodiments, the washout period is at least about 2 weeks (e.g., about 2-6 weeks). In specific embodiments, the washout period is about 2, 3, or 4 weeks. In a specific embodiment, the washout period is about 2 weeks. In a preferred embodiment, the washout period is about 3 weeks. In another specific embodiment, the washout period is about 4 weeks. Preferably, an additional cycle is administered only when the previous cycle has not exhibited toxicity (for example, no grade 3-5 serious adverse events, graded according to NCI CTCAE 4.0).

In specific embodiments, the method of treating comprises administering to the human patient continuously the population of human cells comprising antigen-specific T cells at a dose described herein weekly (i.e., there is no week during which the population of human cells comprising antigen-specific T cells is not administered, and thus there is no washout period).

In certain embodiments, a first dosage regimen described herein is carried out for a first period of time, followed by a second and different dosage regimen described herein that is carried out for a second period of time, wherein the first period of time and the second period of time are optionally separated by a washout period. In specific embodiments, the washout period is at least about 1 week (e.g., about 1-6 weeks). In specific embodiments, the washout period is about 1, 2, 3, or 4 weeks. In specific embodiments, the washout period is at least about 2 weeks (e.g., about 2-6 weeks). In specific embodiments, the washout period is about 2, 3, or 4 weeks. In a specific embodiment, the washout period is about 2 weeks. In a preferred embodiment, the washout period is about 3 weeks. In another specific embodiment, the washout period is about 4 weeks. Preferably, the second dosage regimen is carried out only when the first dosage regimen has not exhibited toxicity (for example, no grade 3-5 serious adverse events, graded according to NCI CTCAE 4.0).

In specific embodiments, the administering of the population of human cells comprising antigen-specific T cells does not result in any graft-versus-host disease (GvHD) in the human patient.

As noted above, the term “about” shall be construed so as to allow normal variation, such as, for example, a variation within 20%.

5.2.2. Serial Treatment with Different Cell Populations

In certain embodiments, the method of treating a disease or disorder associated with a pathogen or treating a cancer in a human patient, as described above, further comprises, after administering to the human patient a first population of human cells comprising antigen-specific T cells that are specific for one or more antigens of the pathogen or cancer, which first population of human cells is derived from a T cell line selected according to a method of selecting a T cell line as described in Section 5.1, administering to the human patient a second population of human cells comprising antigen-specific T cells that are specific for one or more antigens of the pathogen or cancer, which second population of human cells is also derived from a T cell line selected according to a method of selecting a T cell line as described in Section 5.1, wherein the second T cell line is restricted by different HLA alleles (different from the HLA alleles by which the first T cell line is restricted) shared with the human patient (when the human patient has not been the recipient of any cellular transplant) or the Entity (when the human patient has been the recipient of a cellular transplant). In a specific embodiment, the second T cell line is restricted by HLA alleles that are completely different from the HLA alleles by which the first T cell line is restricted. In another specific embodiment, the second T cell line is restricted by HLA alleles that are partially different from the HLA alleles by which the first T cell line is restricted.

In a specific embodiment, the method of treating a disease or disorder associated with a pathogen or treating a cancer in a human patient, as described above, comprises administering a first cycle of one dose per week of the first population of human cells comprising antigen-specific T cells, for at least two consecutive weeks (e.g., 2, 3, 4, 5, or 6 consecutive weeks), optionally followed by a washout period during which no dose of any population of human cells comprising antigen-specific T cells is administered, and followed by a second cycle of one dose per week of the second population of human cells comprising antigen-specific T cells for at least two consecutive weeks (e.g., 2, 3, 4, 5, or 6 consecutive weeks). In specific embodiments, the washout period is at least about 1 week (e.g., about 1-6 weeks). In specific embodiments, the washout period is about 1, 2, 3, or 4 weeks. In specific embodiments, the washout period is at least about 2 weeks (e.g., about 2-6 weeks). In specific embodiments, the washout period is about 2, 3, or 4 weeks. In a specific embodiment, the washout period is about 2 weeks. In a preferred embodiment, the washout period is about 3 weeks. In certain embodiments, the human patient's disease or disorder or the cancer (as the case may be) has no response, an incomplete response, or a suboptimal response (i.e., the human patient may still have a substantial benefit from continuing treatment, but has reduced chances of optimal long-term outcomes) after administering the first population of human cells comprising antigen-specific T cells and prior to administering the second population of human cells comprising antigen-specific T cells.

The first and second populations of human cells comprising antigen-specific T cells can each be administered by any route and any dosage regimen as described in Section 5.2.1, supra.

In a specific embodiment, two populations of human cells comprising antigen-specific T cells each derived from a separate T cell line selected according to a method of selecting a T cell line as described in Section 5.1 and restricted by different HLA alleles shared with the human patient (when the human patient has not been the recipient of any cellular transplant) or the Entity (when the human patient has been the recipient of a cellular transplant) are administered serially. In another specific embodiment, three populations of human cells comprising antigen-specific T cells each derived from a separate T cell line selected according to a method of selecting a T cell line as described in Section 5.1 and restricted by different HLA alleles shared with the human patient (when the human patient has not been the recipient of any cellular transplant) or the Entity (when the human patient has been the recipient of a cellular transplant) are administered serially. In another specific embodiment, four populations of human cells comprising antigen-specific T cells each derived from a separate T cell line selected according to a method of selecting a T cell line as described in Section 5.1 and restricted by different HLA alleles shared with the human patient (when the human patient has not been the recipient of any cellular transplant) or the Entity (when the human patient has been the recipient of a cellular transplant) are administered serially. In another specific embodiment, more than four populations of human cells comprising antigen-specific T cells each derived from a separate T cell line selected according to a method of selecting a T cell line as described in Section 5.1 and restricted by different HLA alleles shared with the human patient (when the human patient has not been the recipient of any cellular transplant) or the Entity (when the human patient has been the recipient of a cellular transplant) are administered serially. In one embodiment, the two, three, four, or more than four populations of human cells comprising antigen-specific T cells described above are each derived from a separate T cell line restricted by completely different HLA alleles shared with the human patient (when the human patient has not been the recipient of any cellular transplant) or the Entity (when the human patient has been the recipient of a cellular transplant). In another embodiment, the two, three, four, or more than four populations of human cells comprising antigen-specific T cells described above are each derived from a separate T cell line restricted by partially different HLA alleles shared with the human patient (when the human patient has not been the recipient of any cellular transplant) or the Entity (when the human patient has been the recipient of a cellular transplant).

5.2.3. Additional Therapies and Previous Therapies

In a specific embodiment, the human patient is concurrently treated with a second therapy for the pathogen-associated disease or disorder, or for the cancer (as the case may be), which second therapy is not treatment with a population of human cells comprising antigen-specific T cells that is derived from a T cell line selected according to this invention, for example, at about the same time, the same day, or same week, or same treatment period (treatment cycle) during which the population of human cells comprising antigen-specific T cells is administered, or on similar dosing schedules, or on different but overlapping dosing schedules. In another specific embodiment, no second therapy for the disease or disorder or the cancer is concurrently administered to the human patient over a period of time over which the population of human cells is repeatedly administered to the human patient. The second therapy can be any therapy known in the art for treating the disease or disorder or the cancer (as the case may be), such as, an antiviral drug therapy (if the disease or disorder is an viral infection), or an anti-cancer therapy (e.g., a chemotherapy, including a combination chemotherapy, or a radiotherapy). In a specific embodiment when the disease or disorder or the cancer to be treated in the human patient is an Epstein-Barr virus (EBV)-associated lymphoproliferative disorder (EBV-LPD) (e.g., an EBV-positive lymphoma), such as an EBV-PTLD, the second therapy is rituximab. In a specific embodiment when the disease or disorder to be treated in the human patient is a cytomegalovirus (CMV) infection, the second therapy is ganciclovir, foscarnet, valganciclovir, cidofovir, leflunomide, or a combination thereof.

In a specific embodiment, the human patient has failed a previous therapy for the pathogen-associated disease or disorder, or for the cancer (as the case may be), which previous therapy is not treatment with a population of human cells comprising antigen-specific T cells that is derived from a T cell line selected according to the invention, due to resistance to or intolerance of the previous therapy. A disease or disorder or a cancer is considered resistant to a therapy, if it has no response, or has an incomplete response (a response that is less than a complete remission), or progresses, or relapses after the therapy. The previous therapy can be any therapy known in the art for treating the disease or disorder or the cancer (as the case may be), such as, an antiviral drug therapy (if the disease or disorder is an viral infection), or an anti-cancer therapy (e.g., a chemotherapy, including a combination chemotherapy, or a radiotherapy). In a specific embodiment when the disease or disorder or the cancer to be treated in the human patient is an EBV-LPD (e.g., an EBV-positive lymphoma), such as an EBV-PTLD, the previous therapy is rituximab. In a specific embodiment when the disease or disorder to be treated in the human patient is a CMV infection, the previous therapy is ganciclovir, foscarnet, valganciclovir, cidofovir, leflunomide, or a combination thereof.

Combination chemotherapy involves the therapeutic use over the same treatment period of two or more different chemotherapeutic agents to treat cancer. Exemplary combination chemotherapies that can be the second therapy or previous therapy described herein include, but are not limited to (the combinations being of the chemotherapeutic agents in parentheses): 7+3 (7 days of cytarabine plus 3 days of an anthracycline antibiotic, either daunorubicin or idarubicin), ABVD (doxorubicin, bleomycin, vinblastine, dacarbazine), BACOD (bleomycin, doxorubicin, cyclophosphamide, vincristine, dexamethasone), BEACOPP (bleomycin, etoposide, doxorubicin, cyclophosphamide, vincristine, procarbazine, prednisone), Dose-Escalated BEACOPP, CBV (cyclophosphamide, carmustine, etoposide), COP (cyclophosphamide, vincristine, and prednisone or prednisolone), CHOEP (cyclophosphamide, doxorubicin, etoposide, vincristine, prednisone), CEOP (cyclophosphamide, etoposide, vincristine, prednisone), CEPP (cyclophosphamide, etoposide, procarbazine, prednisone), ChlVPP (chlorambucil, vincristine, procarbazine, prednisone, etoposide, vinblastine, doxorubicin), CHOP (cyclophosphamide, doxorubicin, vincristine, prednisone), DCEP (dexamethasone, cyclophosphamide, etoposide, platinum agent), DHAP (dexamethasone, cytarabine, platinum agent), DICE (dexamethasone, ifosfamide, cisplatin, etoposide), DT-PACE (dexamethasone, thalidomide, platinum agent, doxorubicin, cyclophosphamide, etoposide), EPOCH (etoposide, prednisone, vincristine, cyclophosphamide, doxorubicin), DA-EPOCH (Dose-Adjusted EPOCH), ESHAP (etoposide, methylprednisolone, cytarabine, cisplatin), FCM (fludarabine, cyclophosphamide, mitoxantrone), FM (fludarabine, mitoxantrone), FLAG (fludarabine, cytarabine, G-CSF), FLAG-IDA (fludarabine, cytarabine, idarubicin, G-CSF), FLAG-MITO (mitoxantrone, fludarabine, cytarabine, G-CSF), FLAMSA (fludarabine, cytarabine, amsacrine), FLAMSA-BU (fludarabine, cytarabine, amsacrine, busulfan), FLAMSA-MEL (fludarabine, cytarabine, amsacrine, melphalan), GVD (gemcitabine, vinorelbine, pegylated liposomal doxorubicin), GEMOX (gemcitabine, oxaliplatin), IAC (idarubicin×3 days plus cytarabine×7 days), ICE (ifosfamide, carboplatin, etoposide), IVAC (etopside, cytarabine, ifosfamide), m-BACOD (methotrexate, bleomycin, doxorubicin, cyclophosphamide, vincristine, dexamethasone), MACOP-B (methotrexate, leucovorin, doxorubicin, cyclophosphamide, vincristine, prednisone, bleomycin), MINE (mesna, ifosfamide, novantrone, etoposide), MOPP (mechlorethamine, vincristine, procarbazine, prednisone), MVP (mitomycin, vindesine, cisplatin), PACE (platinum agent, doxorubicin, cyclophosphamide, etoposide), PEB (cisplatin, etoposide, bleomycin), POMP (6-mercaptopurine, vincristine, methotrexate, prednisone), ProMACE-MOPP (methotrexate, doxorubicin, cyclophosphamide, etoposide, mechlorethamine, vincristine, procarbazine, prednisone), ProMACE-CytaBOM (prednisone, doxorubicin, cyclophosphamide, etoposide, cytarabine, bleomycin, vincristine, methotrexate, leucovorin), RVD (lenalidomide, bortezomib, dexamethasone), Stanford V(doxorubicin, mechlorethamine, bleomycin, vinblastine, vincristine, etoposide, prednisone), Thal/Dex (thalidomide, dexamethasone), VAD (vincristine, doxorubicin, dexamethasone), VAMP (vincristine, amethopterin, 6-mercaptopurine and prednisone, or vincristine, doxorubicin, methotrexate and prednisone, or vincristine, doxorubicin and methylprednisolone), VAPEC-B (vincristine, doxorubicin, prednisone, etoposide, cyclophosphamide, bleomycin), VD-PACE (bortezomib, dexamethasone, platinum agent, doxorubicin, cyclophosphamide, etoposide), VTD-PACE (bortezomib, thalidomide, dexamethasone, platinum agent, doxorubicin, cyclophosphamide, etoposide), DA-REPOCH (rituximab, etoposide, prednisolone, vincristine, cyclophosphamide, hydroxydaunorubicin), RIVAC (rituximab, ifosphamide, etoposide, cytarabine), and RGDP (rituximab, gemcitabine, dexamethasone, cisplatin).

Radiation therapies use high-energy radiation to kill cancer cells by damaging their DNA. Exemplary radiation therapies that can be the second therapy or previous therapy described herein include, but are not limited to conventional external beam radiation therapy, stereotactic radiation therapy, intensity-modulated radiation therapy, volumetric modulated arc therapy, particle therapy, auger therapy, brachytherapy, and radioisotope therapy.

5.3. Generation of T Cell Lines and Populations of Human Cells Comprising Antigen-Specific T Cells

The collection of T cell lines used in the methods of selecting that are disclosed herein can be selected from among those available in the art or made by methods described herein. In a specific embodiment, the collection of T cell lines used is a bank of cryopreserved T cell lines. In specific embodiments, the collection of T cell lines contains at least 10, 50, 100 or 200 different T cell lines. The T cell lines in the collection of T cell lines described in this disclosure (including the T cell line selected for therapeutic administration to the human patient) and the population of human cells comprising antigen-specific T cells derived from the selected T cell line can be generated as described herein. Preferably, all of the T cell lines in the collection of T cell lines are generated using the same method.

5.3.1. Ex Vivo Sensitization

In some embodiments, a T cell line described herein is generated by ex vivo sensitizing human T cells to one or more antigens of the pathogen or cancer, said ex vivo sensitizing comprises co-culturing, over a period of time in culture, a population of human blood cells comprising the human T cells with antigen presenting cells presenting the one or more antigens. In a preferred embodiment, the ex vivo sensitizing results in expansion of antigen-specific T cells that are specific for the one or more antigens. In a specific embodiment, the human T cells that are ex vivo sensitized are not genetically engineered to be specific for the one or more antigens (e.g., by expression of a chimeric antigen receptor (CAR) or T cell receptor (TCR) specific to the one or more antigens). In a specific embodiment, the human T cells that are ex vivo sensitized are genetically engineered other than for antigen specificity (for example, to express a pro-immune response cytokine).

The ex vivo sensitizing step can be performed by any method known in the art to stimulate T cells to be antigen-specific ex vivo, such as a method as described in Section 6 (Example); Koehne et al., 2000, Blood 96:109-117; Trivedi et al., 2005, Blood 105:2793-2801; Hague et al., 2007, Blood 110:1123-1131; Hasan et al., 2009, J Immunol 183: 2837-2850; Feuchtinger et al., 2010, Blood 116:4360-4367; Doubrovina et al., 2012, Blood 120:1633-1646; Leen et al., 2013, Blood 121:5113-5123; Papadopoulou et al., 2014, Sci Transl Med 6:242ra83; Sukdolak et al., 2013, Biol Blood Marrow Transplant 19:1480-1492; Koehne et al., 2015, Biol Blood Marrow Transplant 21: 1663-1678; International Patent Application Publication No. WO 2016/073550; International Patent Application Publication No. WO 2016/183153; International Patent Application Publication No. WO 2016/209816; or International Patent Application Publication No. WO 2017/044678.

In specific embodiments, the aforementioned period of time in culture (termed herein “the Sensitization Culture Time;” i.e., the culture time period over which co-culturing occurs) is at least 7 days. In specific embodiments, the Sensitization Culture Time is at least 14 days. In specific embodiments, the Sensitization Culture Time is at least 21 days. In specific embodiments, the Sensitization Culture Time is at least 28 days. In specific embodiments, the Sensitization Culture Time is in the range of 21-28 days. In specific embodiments, the Sensitization Culture Time is in the range of 28-35 days. In a specific embodiment, the Sensitization Culture Time is 21 days. In another specific embodiment, the Sensitization Culture Time is 22 days. In another specific embodiment, the Sensitization Culture Time is 23 days. In another specific embodiment, the Sensitization Culture Time is 24 days. In another specific embodiment, the Sensitization Culture Time is 25 days. In another specific embodiment, the Sensitization Culture Time is 26 days. In another specific embodiment, the Sensitization Culture Time is 27 days. In a preferred embodiment, the Sensitization Culture Time is 28 days. In another specific embodiment, the Sensitization Culture Time is 29 days. In another specific embodiment, the Sensitization Culture Time is 30 days. In another specific embodiment, the Sensitization Culture Time is 31 days. In another specific embodiment, the Sensitization Culture Time is 32 days. In another specific embodiment, the Sensitization Culture Time is 33 days. In another specific embodiment, the Sensitization Culture Time is 34 days. In another specific embodiment, the Sensitization Culture Time is 34 days.

The antigen presenting cells used in the ex vivo sensitizing step can be any antigen presenting cells suitable for presenting the one or more antigens, including professional antigen presenting cells and non-professional antigen presenting cells, and are typically irradiated cells to prevent multiplication of these cells after being added to the culture. In specific embodiments, the antigen presenting cells used in the ex vivo sensitizing step are dendritic cells, cytokine-activated monocytes, peripheral blood mononuclear cells (PBMCs), Epstein-Barr virus-transformed B-lymphoblastoid cell line cells (EBV-BLCL cells), or artificial antigen presenting cells (AAPCs). In a specific embodiment, the antigen presenting cells are dendritic cells. In another specific embodiment, the antigen presenting cells are PBMCs. In another specific embodiment, the antigen presenting cells are EBV-BLCL cells. In another specific embodiment, the antigen presenting cells are AAPCs. In some embodiments, the antigen presenting cells are derived from the donor of the population of human blood cells. In other embodiments, the antigen presenting cells are allogeneic to the donor of the population of human blood cells. The antigen presenting cells can be obtained by any method known in the art, such as the method(s) described in Section 6 (Example); Koehne et al., 2000, Blood 96:109-117; Koehne et al., 2002, Blood 99:1730-1740; Trivedi et al., 2005, Blood 105:2793-2801; O'Reilly et al., 2007, Immunol Res 38:237-250; Hasan et al., 2009, J Immunol 183: 2837-2850; Barker et al., 2010, Blood 116:5045-5049; 0′ Reilly et al., 2011, Best Practice & Research Clinical Haematology 24:381-391; Doubrovina et al., 2012, Blood 120:1633-1646; Koehne et al., 2015, Biol Blood Marrow Transplant 21: 1663-1678; International Patent Application Publication No. WO 2016/073550; International Patent Application Publication No. WO 2016/183153; International Patent Application Publication No. WO 2016/209816; or International Patent Application Publication No. WO 2017/044678.

In various embodiments, the ex vivo sensitizing further comprises adding antigen presenting cells presenting the one or more antigens to the culture. In specific embodiments, the ex vivo sensitizing further comprises adding antigen presenting cells presenting the one or more antigens to the culture at the initiation of said co-culturing and every 1 to 14 days thereafter during the co-culturing. In specific embodiments, the ex vivo sensitizing further comprises adding antigen presenting cells presenting the one or more antigens to the culture at the initiation of said co-culturing and every 3 to 12 days thereafter during the co-culturing. In specific embodiments, the ex vivo sensitizing further comprises adding antigen presenting cells presenting the one or more antigens to the culture at the initiation of said co-culturing and every 5 to 10 days thereafter during the co-culturing. In preferred embodiments, the ex vivo sensitizing further comprises adding antigen presenting cells presenting the one or more antigens to the culture at the initiation of said co-culturing and every 7 to 10 days thereafter during the co-culturing. In a specific embodiment, the ex vivo sensitizing further comprises adding antigen presenting cells presenting the one or more antigens to the culture at the initiation of said co-culturing and about every 5 days thereafter during the co-culturing. In another specific embodiment, the ex vivo sensitizing further comprises adding antigen presenting cells presenting the one or more antigens to the culture at the initiation of said co-culturing and about every 6 days thereafter during the co-culturing. In another specific embodiment, the ex vivo sensitizing further comprises adding antigen presenting cells presenting the one or more antigens to the culture at the initiation of said co-culturing and about every 7 days thereafter during the co-culturing. In another specific embodiment, the ex vivo sensitizing further comprises adding antigen presenting cells presenting the one or more antigens to the culture at the initiation of said co-culturing and about every 8 days thereafter during the co-culturing. In another specific embodiment, the ex vivo sensitizing further comprises adding antigen presenting cells presenting the one or more antigens to the culture at the initiation of said co-culturing and about every 9 days thereafter during the co-culturing. In another specific embodiment, the ex vivo sensitizing further comprises adding antigen presenting cells presenting the one or more antigens to the culture at the initiation of said co-culturing and about every 10 days thereafter during the co-culturing.

In some embodiments, the antigen presenting cells are loaded with one or more immunogenic peptides or proteins derived from the one or more antigens. Non-limiting exemplary methods for loading antigen presenting cells with peptide(s) derived from antigen(s) can be found in Trivedi et al., 2005, Blood 105:2793-2801; Barker et al., 2010, Blood 116:5045-5049; Doubrovina et al., 2012, Blood 120:1633-1646; Hasan et al., 2009, J Immunol 183: 2837-2850; Koehne et al., 2015, Biol Blood Marrow Transplant 21: 1663-1678; International Patent Application Publication No. WO 2016/073550; International Patent Application Publication No. WO 2016/183153; International Patent Application Publication No. WO 2016/209816; and International Patent Application Publication No. WO 2017/044678. In other embodiments, the antigen presenting cells are genetically engineered to recombinantly express one or more immunogenic peptides or proteins derived from the one or more antigens. Any appropriate method known in the art for introducing nucleic acid vehicles into cells to express proteins, such as transduction or transformation, can be used to genetically engineer the antigen presenting calls to recombinantly express the one or more immunogenic peptides or proteins derived from the one or more antigens.

In some embodiments, the one or more immunogenic peptides or proteins are a pool of overlapping peptides derived from the one or more antigens. In specific embodiments, the pool of overlapping peptides is a pool of overlapping pentadecapeptides. In other embodiments, the one or more immunogenic peptides or proteins are one or more proteins derived from the one or more antigens.

In specific embodiments, the method of generating a T cell line described herein further comprises, after the step of ex vivo sensitizing, a step of cryopreserving the ex vivo sensitized (and preferably expanded) human T cells, or a fraction thereof.

The population of human cells comprising antigen-specific T cells that is derived from the selected T cell line can be generated by, for example, taking the whole or a fraction of the selected T cell line (which is optionally expanded in culture), or thawing (and optionally expanding in culture) a cryopreserved selected T cell line or a fraction thereof.

The cryopreserving and thawing described herein can be performed by known methods in the art for cryopreserving T cells and thawing T cells, respectively.

The term “about” shall be construed so as to allow normal variation, such as, for example, a variation within 20%.

5.3.2. Purification from a Blood-Derived Sample

In other embodiments, a T cell line described herein is derived from (for example, expanded from) antigen-specific T cells purified from a population of human blood cells (such as peripheral blood mononuclear cells (PBMCs)) that is seropositive for the one or more antigens (for example, by sorting (such as fluorescence activated cell sorting) T cells that recognize the one or more antigens from the blood sample cells). In a specific embodiment, the antigen-specific T cells purified from the population of human blood cells and the T cells contained in the T cell line are not genetically engineered to be specific for the one or more antigens (e.g., by expression of a chimeric antigen receptor (CAR) or T cell receptor (TCR) specific to the one or more antigens). In a specific embodiment, the antigen-specific T cells purified from the population of human blood cells and the T cells contained in the T cell line are genetically engineered other than for antigen specificity (for example, to express a pro-immune response cytokine).

The population of human cells comprising antigen-specific T cells that is derived from the selected T cell line can be generated by, for example, taking the whole or a fraction of the selected T cell line (which is optionally expanded in culture), or thawing (and optionally expanding in culture) a cryopreserved selected T cell line or a fraction thereof.

The cryopreservation and thawing described herein can be performed by known methods in the art for cryopreserving T cells and thawing T cells, respectively.

5.3.3. The Population of Human Blood Cells Used for Generating T Cell Line

The population of human blood cells used for generating a T cell line, as described in Sections 5.3.1-5.3.2, can be any cell sample that contains T cells, such as, but is not limited to, a hematopoietic cell sample, a fractionated or unfractionated whole blood sample, a fractionated or unfractionated apheresis collection (e.g., a leukapheresis collection, such as leukopak), PBMCs, or a purified T cell population (e.g., T cells enriched from PBMCs). In a specific embodiment, the population of human blood cells is a population of human PBMCs. PBMCs can be isolated by any method known in the art to isolate PBMCs from a blood sample, such as by Ficoll-Hypaque centrifugation as described in Koehne et al., 2000, Blood 96:109-117 or Trivedi et al., 2005, Blood 105:2793-2801. In another specific embodiment, the population of human blood cells is a population enriched in T cells from PBMCs. T cells can be enriched for from the PBMCs by any method known in the art to enrich for T cells from a blood sample or PBMCs. Non-limiting exemplary methods for enriching for T cells from PBMCs can be found in Koehne et al., 2000, Blood 96:109-117; Trivedi et al., 2005, Blood 105:2793-2801; Hasan et al., 2009, J Immunol 183: 2837-2850; and Koehne et al., 2015, Biol Blood Marrow Transplant 21: 1663-1678. For example, T cells can be enriched for from PBMCs by sorting the PBMCs using an anti-CD3 antibody and/or depleting from the PBMCs adherent monocytes and natural killer cells.

In preferred embodiments, the population of human blood cells is derived from a human donor that is seropositive for the one or more antigens. In certain embodiments, the population of human blood cells is derived from a human donor that is seronegative for the one or more antigens.

In some embodiments, the population of human blood cells (thus, the T cell line) is derived autologously from the human patient. In other embodiments, the population of human blood cells (thus, the T cell line) is derived from a human donor that is allogeneic to the human patient. In a specific embodiment, the human patient has been the recipient of a cellular transplant from a donor of the cellular transplant, and the human donor from whom the population of human blood cells (thus, the T cell line) is derived is a third-party donor that is different from the donor of the cellular transplant. In another specific embodiment, the human patient has been the recipient of a cellular transplant from a donor of the cellular transplant, and the human donor from whom the population of human blood cells (thus, the T cell line) is derived is the donor of the cellular transplant. The cellular transplant can be a hematopoietic stem cell transplant (HSCT) (such as a peripheral blood stem cell transplant, a bone marrow transplant, or a cord blood transplant), a tissue transplant (such as a skin transplant, a bone transplant, a tendon transplant, a cornea transplant, a heart valve transplant, a nerve transplant, or a vein transplant), or a solid organ transplant (such as a kidney transplant, a liver transplant, a heart transplant, an intestinal transplant, a pancreas transplant, a lung transplant, or a small bowel transplant).

The human donor from whom the population of human blood cells (thus, the T cell line) is derived can be an adult (at least age 16), an adolescent (age 12-15), a child (under age 12), a fetus, or a neonate. In a specific embodiment, the human donor from whom the population of human blood cells (thus, the T cell line) is derived is an adult. In a specific embodiment, the population of human blood cells (thus, the T cell line) is derived from human (umbilical) cord blood.

In specific embodiments, the population of human blood cells used for generating a T cell line described herein comprises CD4⁺ T cells. In specific embodiments, the population of human blood cells used for generating a T cell line described herein comprises CD8⁺ T cells. In a specific embodiment, the population of human blood cells used for generating a T cell line described herein comprises both CD4⁺ and CD8⁺ T cells.

In a specific embodiment, the population of human blood cells used for generating a T cell line described herein contains at least 50% T cells. In another specific embodiment, the population of human blood cells contains at least 60% T cells. In another specific embodiment, the population of human blood cells contains at least 70% T cells. In a specific embodiment, the population of human blood cells contains at least 80% T cells. In a specific embodiment, the population of human blood cells contains at least 90% T cells. In a specific embodiment, the population of human blood cells contains at least 95% T cells. In a specific embodiment, the population of human blood cells contains at least 99% T cells. In a specific embodiment, the population of human blood cells contains 100% T cells.

In certain embodiments, the population of human blood cells used for generating a T cell line described herein contains, at initiation of generation, at least 50% memory T cells. In a specific embodiment, the population of human blood cells used for generating a T cell line described herein contains, at initiation of generation, at least 60% memory T cells. In another specific embodiment, the population of human blood cells used for generating a T cell line described herein contains, at initiation of generation, at least 70% memory T cells. In another specific embodiment, the population of human blood cells used for generating a T cell line described herein contains, at initiation of generation, at least 80% memory T cells. In another specific embodiment, the population of human blood cells used for generating a T cell line described herein contains, at initiation of generation, at least 90% memory T cells. In another specific embodiment, the population of human blood cells used for generating a T cell line described herein contains, at initiation of generation, at least 95% memory T cells. In another specific embodiment, the population of human blood cells used for generating a T cell line described herein contains, at initiation of generation, at least 99% memory T cells. In another specific embodiment, the population of human blood cells used for generating a T cell line described herein contains, at initiation of generation, 100% memory T cells. The memory T cells described herein can be central memory T cells (T_{CM} cells), stem cell-like memory T cells (T_{SCM} cells), effector memory T cells (T_{EM} cells), or a combination thereof.

5.3.4. Storage

A T cell line described herein (either the selected T cell line or a non-selected T cell line form the collection of T cell lines) can be stored in a pharmaceutical composition with a pharmaceutically acceptable carrier.

The pharmaceutical acceptable carrier can be any physiologically-acceptable solution suitable for the storage and/or therapeutic administration of T cells, for example, a saline solution, a buffered saline solution, or a bio-compatible solution comprising one or more cryopreservatives (e.g., phosphate-buffered saline containing 7% DMSO, 5% dextrose and 1% dextran; hypothermosol containing 5% DMSO and 5% human serum albumin; normal saline containing 10% DMSO and 16% human serum albumin; or normal saline containing 10% DMSO and 15% human serum albumin).

A T cell line can be stored in the pharmaceutical composition at any concentration desirable for its long-term storage and convenience of storage and handling. In a specific embodiment, the T cell line is stored in the pharmaceutical composition at a concentration of about 5×10⁶ cells/ml. In another specific embodiment, the T cell line is stored in the pharmaceutical composition at a concentration of about 10×10⁶ cells/ml. In another specific embodiment, the T cell line is stored in the pharmaceutical composition at a concentration of about 20×10⁶ cells/ml. In another specific embodiment, the T cell line is stored in the pharmaceutical composition at a concentration of about 50×10⁶ cells/ml. In another specific embodiment, the T cell line is stored in the pharmaceutical composition at a concentration of about 100×10⁶ cells/ml. In another specific embodiment, the T cell line is stored in the pharmaceutical composition at a concentration of about 200×10⁶ cells/ml. In another specific embodiment, the T cell line is stored in the pharmaceutical composition at a concentration of about 500×10⁶ cells/ml. In another specific embodiment, the T cell line is stored in the pharmaceutical composition at a concentration of about 1 to 10×10⁶ cells/ml. In another specific embodiment, the T cell line is stored in the pharmaceutical composition at a concentration of about 10 to 100×10⁶ cells/ml. In another specific embodiment, the T cell line is stored in the pharmaceutical composition at a concentration of about 100 to 1000×10⁶ cell s/ml.

In a specific embodiment, the pharmaceutical composition is stored in a cryopreserved form before preparation for administration to the human patient of the population of human cells comprising antigen-specific T cells derived from the selected T cell line contained in the pharmaceutical composition. For example, the pharmaceutical composition can be stored at a temperature of −150° C. or less, until just prior to preparation for administration. To prepare for intravenous administration, the cryopreserved pharmaceutical composition is thawed and optionally diluted in a sterile, nonpyrogenic isotonic solution (for example, Normosol® or PlasmaLyte®) to a final volume of up to 50 ml.

Also described herein are kits comprising in one or more containers the pharmaceutical composition described herein. In specific embodiments, the kits further comprise a second pharmaceutical composition comprising a second compound or biological product for treating the pathogen or cancer.

Optionally associated with such one or more containers can be a notice in the form prescribed by a governmental agency regulating the manufacture, use or sale of pharmaceuticals or biological products, which notice reflects approval by the agency of manufacture, use or sale for human administration.

The pharmaceutical compositions and kits described herein can be used in accordance with the methods of treating a disease or disorder associated with a pathogen or treating a cancer in a human patient as provided in this disclosure.

As stated above, the term “about” shall be construed so as to allow normal variation, such as, for example, a variation within 20%.

5.4. Computer Systems and Computer Readable Media

In various embodiments, a computer system or computer readable medium is configured for carrying out any of the methods of selecting a T cell line as described in this disclosure, and then preferably outputting the selected T cell line.

Also provided herein are computer systems for selecting a T cell line for therapeutic administration to a human patient to treat a disease or disorder associated with a pathogen or to treat a cancer in the human patient. In a specific embodiment such a computer system comprises: a central processing unit; a memory, coupled to the central processing unit, the memory storing instructions for performing the step(s) of any of the methods of selecting a T cell line as described in this disclosure. In a specific embodiment, the memory of the computer system also stores instructions for a step of outputting the selected T cell line.

In some embodiments, the computer system further comprises a display device in operable communication with the central processing unit.

Also provided herein are computer readable media having computer-executable instructions for performing the step(s) of any of the methods of selecting a T cell line as described in this disclosure, and then preferably outputting the selected T cell line.

In some embodiments, loaded into a computer system or computer readable medium are software components that are standard in the art. The software components collectively cause the computer system to function according to a method of selecting a T cell line as described in this disclosure. In some embodiments, loaded into the computer system or computer readable medium are software components that are standard in the art, and one or more computer program products that are special to the instant invention. In specific embodiments, the one or more computer program products cause a computer system to function according to a method of selecting a T cell line as described in this disclosure, and preferably then to output the selected T cell line. In specific embodiments, the one or more computer program products that are special to the instant invention and the software components that are standard in the art collectively cause the computer system to function according to a method of selecting a T cell line as described herein.

5.5. Antigen Specificity and Patients

According to the invention, the T cell line selected according to a method described in Section 5.1 for therapeutic administration to a human patient to treat a disease or disorder associated with a pathogen or to treat a cancer in the human patient exhibits a T cell response against one or more antigens of the pathogen or cancer.

The one or more antigens of a pathogen or cancer can be one or more peptides or proteins whose respective expression is higher in the diseased cells that express the one or more antigens of the pathogen or cancer relative to non-diseased cells (typically of the same tissue type as the diseased cells) (for example, cells not infected by the pathogen, or non-cancerous cells) or unique in the diseased cells that express the one or more antigens of the pathogen or cancer relative to non-diseased cells (typically of the same tissue type as the diseased cells) (for example, cells not infected by the pathogen, or non-cancerous cells).

In some embodiments, the method of selecting a T cell line described herein is of selecting a T cell line for therapeutic administration to the human patient to treat a disease or disorder associated with a pathogen, and the one or more antigens are one or more antigens of a pathogen. A “disease or disorder associated with a pathogen” as used herein refers to a disease or disorder that results from the presence of the pathogen, and can be, as non-limiting examples, a pathogen-positive cancer, viremia, or an infection by the pathogen. In a specific embodiment, the disease or disorder associated with a pathogen is an infection by the pathogen. In a further specific embodiment, the disease or disorder associated with a pathogen is an active infection by the pathogen.

The pathogen can be a virus, bacterium, fungus, helminth or protist.

In specific embodiments, the pathogen is a virus. In a specific embodiment, the virus is cytomegalovirus (CMV). In an aspect of the specific embodiment, the one or more antigens of CMV is CMV pp65, CMV IEL or a combination thereof. In another aspect of the specific embodiment, the one or more antigens of CMV is CMV pp65. In another specific embodiment, the virus is Epstein-Barr virus (EBV). In an aspect of the specific embodiment, the one or more antigens of EBV is EBNA1, EBNA2, EBNA3A, EBNA3B, EBNA3C, LMP1, LMP2, or a combination thereof. In another aspect of the specific embodiment, the one or more antigens of EBV is EBNA1, LMP1, LMP2, or a combination thereof. In another specific embodiment, the virus is BK virus (BKV), John Cunningham virus (JCV), herpesvirus (such as human herpesvirus-6 or human herpesvirus-8), human papillomavirus (HPV), hepatitis B virus (HBV), hepatitis C virus (HCV), herpes simplex virus (HSV), varicella zoster virus (VZV), Merkel cell polyomavirus (MCV), adenovirus (ADV), human immunodeficiency virus (HIV), influenza virus, ebola virus, poxvirus, rhabdovirus, or paramyxovirus.

In specific embodiments, the pathogen is a bacterium, such as a mycobacterium or Chlamydia trachomatis. In specific embodiments, the pathogen is a fungus, such as Cryptococcus neoformans, Pneumocystis jiroveci, a Candida, or an invasive fungus. In specific embodiments, the pathogen is a helminth. In specific embodiments, the pathogen is a protist, such as Toxoplasma gondii. In specific embodiments, the pathogen is a protozoa.

In a specific embodiment, the pathogen is CMV and the disease or disorder associated with the pathogen is a CMV infection (e.g., CMV viremia, CMV retinitis, CMV pneumonia, CMV hepatitis, CMV colitis, CMV encephalitis, CMV meningoencephalitis, CMV-positive meningioma, or CMV-positive glioblastoma multiforme). In another specific embodiment, the pathogen is EBV and the disease or disorder associated with an antigen is an EBV-positive lymphoproliferative disorder (EBV-LPD) (for example, an EBV-PTLD) resulting from EBV infection, such as B-cell hyperplasia, lymphoma (such as, B-cell lymphoma, non-Hodgkin lymphoma (e.g., diffuse large B-cell lymphoma, for example in the elderly), T-cell lymphoma, EBV-positive Hodgkin's lymphoma, Burkitt lymphoma), polymorphic or monomorphic EBV-LPD, autoimmune lymphoproliferative syndrome, or mixed post-transplant lymphoproliferative disorder (PTLD). In another specific embodiment, the pathogen is EBV and the disease or disorder associated with the pathogen is an EBV-positive nasopharyngeal carcinoma. In another specific embodiment, the pathogen is EBV and the disease or disorder associated with the pathogen an EBV-positive gastric cancer. In another specific embodiment, the pathogen is EBV and the disease or disorder associated with the pathogen is an EBV-positive leiomyosarcoma. In another specific embodiment, the pathogen is EBV and the disease or disorder associated with the pathogen is an EBV-positive NK/T lymphoma. In another specific embodiment, the pathogen is EBV and the disease or disorder associated with the pathogen is an EBV viremia.

In other embodiments, the method of selecting a T cell line described herein is of selecting a T cell line for therapeutic administration to a human patient to treat a cancer in the human patient, and the one or more antigens are one or more antigens of a cancer.

The cancer can be a blood cancer, such as, but is not limited to: acute lymphoblastic leukemia, chronic lymphocytic leukemia, acute myelogenous leukemia, chronic myelogenous leukemia, hairy cell leukemia, T-cell prolymphocytic leukemia, Large granular lymphocytic leukemia, adult T-cell leukemia, plasma cell leukemia, Hodgkin lymphoma, Non-Hodgkin lymphoma, or multiple myeloma. In specific embodiments, the cancer is a lymphoproliferative disorder (LPD). In a specific embodiment, the LPD is a lymphoma, such as, for example, a B-cell lymphoma, a T-cell lymphoma, an NK/T lymphoma, a Burkitt lymphoma, a Hodgkin lymphoma or a Non-Hodgkin lymphoma. In a specific embodiment, the lymphoma is diffuse large B-cell lymphoma (DLBCL) (for example, a non-germinal center B cell-like DLBCL). In another specific embodiment, the lymphoma is plasmablastic lymphoma (PBL).

The cancer can also be a solid tumor cancer, including, but is not limited to, a sarcoma, a carcinoma, a lymphoma, a germ cell tumor, or a blastoma. The solid tumor cancer can be, such as, but is not limited to: a cancer of the breast, lung, ovary, stomach, pancreas, larynx, esophagus, testes, liver, parotid, biliary tract, colon, rectum, cervix, uterus, endometrium, kidney, bladder, prostate, thyroid, brain, or skin.

In a specific embodiment, the one or more antigens of the cancer is Wilms Tumor 1 (WT1). In an aspect of the specific embodiment, the cancer is multiple myeloma or plasma cell leukemia. In an aspect of the specific embodiment, the human patient has multiple myeloma or plasma cell leukemia.

In another specific embodiment, the one or more antigens of the cancer are one or more antigens of EBV, such as, for example, EBNA1, EBNA2, EBNA3A, EBNA3B, EBNA3C, LMP1, and/or LMP2. In a specific embodiment, the cancer is an EBV-positive LPD (e.g., an EBV-positive lymphoma), such as an EBV-PTLD, and the one or more antigens are one or more antigens of EBV, such as, for example, EBNA1, EBNA2, EBNA3A, EBNA3B, EBNA3C, LMP1, and/or LMP2. In another specific embodiment, the cancer is an EBV-positive nasopharyngeal carcinoma and the one or more antigens are one or more antigens of EBV, such as, for example, EBNA1, EBNA2, EBNA3A, EBNA3B, EBNA3C, LMP1, and/or LMP2. In another specific embodiment, the cancer is an EBV-positive gastric cancer and the one or more antigens are one or more antigens of EBV, such as, for example, EBNA1, EBNA2, EBNA3A, EBNA3B, EBNA3C, LMP1, and/or LMP2. In another specific embodiment, the cancer is an EBV-positive leiomyosarcoma and the one or more antigens are one or more antigens of EBV, such as, for example, EBNA1, EBNA2, EBNA3A, EBNA3B, EBNA3C, LMP1, and/or LMP2.

In another specific embodiment the one or more antigens of the cancer are one or more antigens of CMV, such as CMV pp65 and/or CMV IE1. In a specific embodiment, the cancer is CMV-positive glioblastoma multiforme and the one or more antigens are one or more antigens of CMV, such as CMV pp65 and/or CMV 1E1.

In various embodiments as described in this disclosure, the human patient has been immunocompromised.

In a specific embodiment as described in this disclosure, the human patient has been the recipient of a cellular transplant. In some embodiments, the human patient has been the recipient of a solid organ transplant. The solid organ transplant can be, but is not limited to, a kidney transplant, a liver transplant, a heart transplant, an intestinal transplant, a pancreas transplant, a lung transplant, a small bowl transplant, or a combination thereof. In other embodiments, the human patient has been the recipient of a hematopoietic stem cell transplant (HSCT), for example, a T-cell depleted HSCT. The HSCT can be a bone marrow transplant, a peripheral blood stem cell transplant, or a cord blood transplant. In other embodiments, the human patient has been the recipient of a tissue transplant, for example, a skin transplant, a bone transplant, a tendon transplant, a cornea transplant, a heart valve transplant, a nerve transplant, or a vein transplant.

In another specific embodiment as described in this disclosure, the human patient is not the recipient of any cellular transplant.

In another specific embodiment as described in this disclosure, the human patient is HIV-infected. In a particular embodiment, the human patient has acquired immunodeficiency syndrome (AIDS).

In another specific embodiment as described in this disclosure, the human patient has received immunosuppressant therapy (for example, after solid organ transplant).

In another specific embodiment as described in this disclosure, the human patient has a primary immunodeficiency (for example, a genetic disorder that has caused immunodeficiency).

In a specific embodiment as described in this disclosure, the human patient is an adult (at least age 16). In another specific embodiment as described in this disclosure, the human patient is an adolescent (age 12-15). In another specific embodiment as described in this disclosure, the patient is a child (under age 12).

### 6. EXAMPLE

Certain embodiments provided herein are illustrated by the following non-limiting example.

6.1. Summary

Adoptive transfer of donor-derived EBV-specific T-cells (EBV-CTLs) can eradicate EBV associated lymphomas post hematopoietic cell (HCT) or solid organ (SOT) transplants but is not available for most patients. This study developed a 3rd party, allogeneic, off-the-shelf bank of 330 GMP grade EBV-CTL lines from specifically consented healthy HCT donors. 46 recipients of HCT (N=33) or SOT (N=13) with biopsy-proven EBV associated lymphomas who failed rituximab therapy were treated with 3rd party EBV-CTLs. Treatment cycles consisted of 3 weekly infusions of EBV-CTLs and 3 weeks of observation. The EBV-CTLs did not induce significant toxicities or graft injury. One patient developed grade I skin GVHD. Complete and sustained partial remissions were achieved in 68% of HCT recipients and 54% of SOT recipients. For patients who achieved CR/PR or stable disease after cycle 1, overall survival without recurrence was 89.9% and 81.8% respectively at 1 year. Although only 1/11 patients (9.1%) with progression of disease (POD) after cycle 1 who received additional EBV-CTLs from the same donor survived, 3 of 5 with POD subsequently treated with EBV-CTLs from a different donor achieved CR or durable PR (60%) and survive >1 year. Maximal responses were achieved after a median of 2 cycles. Third party EBV-CTLs of defined HLA restriction provide safe, immediately accessible treatment for EBV PTLD. Secondary treatment with EBV-CTLs restricted by a different HLA allele (switch therapy) can also induce remissions if initial EBV-CTLs are ineffective. These results suggest a promising potential therapy for patients with rituximab refractory EBV-associated lymphoma post transplant.

6.2. Introduction

EBV-induced malignancies are a significant cause of morbidity and mortality for recipients of allogeneic hematopoietic cell transplants (HCT), solid organ transplant (SOT) and immunocompromised patients (Singavi et al., 2015, Cancer Treatment and Research 165:305-327). In the population of HCT recipients, those receiving T cell-depleted grafts and those who receive antithymocyte globulin (ATG) particularly with cord blood grafts are at especially high risk (Hoegh-Petersen et al., 2011, Bone Marrow Transplantation 46:1104-1112; and Hoegh-Petersen et al., 2011, Bone Marrow Transplantation 46:1104-1112). High risk SOT patients include recipients who are seronegative pre-transplant and recipients of lung, heart and small intestinal transplants. EBV driven PTLD typically ranges from “benign” hyperplastic lesions to rapidly progressive, monoclonal, diffuse large B-cell lymphoma (DLBCL) (Morscio et al., 2013, Clinical and Developmental Immunology Article ID 150835; and Petrara et al., 2015, Cancer Letters 369:37-44).

Decreasing immune suppression can induce remissions of EBV-PTLD in recipients of SOT with early polyclonal proliferations (Petrara et al., 2015, Cancer Letters 369:37-44) but is not effective in recipients of hematopoietic transplants or in SOT recipients with more aggressive, monoclonal/monomorphic disease (Petrara et al., 2015, Cancer Letters 369:37-44) and can be associated with graft rejection in up to 15% of patients (Franke et al., 2017, Hematological Oncology, 35(Suppl S2):349 (abstract 389)). While combination chemotherapy can induce remissions in 40%-50% of SOT patients with monoclonal disease, relapses are common (Glotz et al., 2012, Transplantation 94:784-793; and Trappe et al., 2012, The Lancet Oncology 13:196-206). In HCT patients, combination chemotherapy is associated with increased morbidity and mortality (Davis, 2001, Transplant Infectious Disease 3:108-118). Similarly, in recipients of SOT treatment related mortality after R-CHOP ranges from 6-30%. The CD20-specific mAb rituximab administered preemptively can induce sustained reversal of EBV viremia in up to 83% of recipients (Styczynski et al., 2013, Clinical Infectious Diseases 57:794-802); but only 50%-60% of patients with clinically and radiologically established disease achieve remissions (Choquet et al., 2007, Annals of Hematology 86:599-607; Trappe et al., 2016, Journal of Clinical Oncology 35:536-543; and Fox et al., 2014, Bone Marrow Transplantation 49:280-286).

In 1994, the O'Reilly group at Memorial Sloan Kettering Cancer Center (MSKCC) first reported 5 HCT patients with monoclonal EBV-associated lymphomas who achieved durable complete remission (CR) after adoptive transfer of donor leukocytes (DLI) containing unselected T cells from their EBV-seropositive transplant donors and correlated these responses with the emergence of donor-derived EBV-CTLs in the blood post transfer (Papadopoulos et al., 1994, The New England Journal of Medicine 330:1185-1191; and Lucas et al., 1996, Blood 87:2594-2603). In 1995, Rooney et al first (Rooney et al., 1995, Lancet 345:9-13) reported the use of HCT donor-derived EBV-specific cytotoxic T cells (EBV-CTLs) generated in vitro to reconstitute EBV-specific immunity and treat or prevent EBV lymphomas following transplant without graft versus host disease (GvHD) (Heslop et al., 1996, Nature Medicine 2:551-555). Subsequently, several groups, including the O'Reilly group, have reported case series demonstrating that adoptive transfer of HCT donor-derived EBV-CTLs can induce clearance of viremia and durable complete remissions of biopsy proven monoclonal lymphomas in 50-70% of cases, including patients failing treatment with Rituximab alone or in combination with chemotherapy (Doubrovina et al., 2012, Blood 119:2644-2656; Gerdemann et al., 2012, Molecular Therapy 20:1622-1632; Icheva et al., 2013, Journal of Clinical Oncology 31:39-48; Comoli et al., 2007, American Journal of Transplantation 7:1648-1655; and Imashuku et al., 1997, Bone Marrow Transplantation 20:337-340). For SOT patients, case reports have documented that autologous EBV-CTLs generated in vitro can also induce complete or transient partial clinical remissions of EBV lymphomas (Savoldo et al., 2006, Blood 108:2942-2949; Khanna et al., 1999, Proceedings of the National Academy of Sciences of the United States of America 96:10391-10396; Comoli et al., 2005, American Journal of Transplantation 5:1415-1422; and Sherritt et al., 2003, Transplantation 75:1556-1560). However, EBV viremia is rarely cleared (Khanna et al., 1999, Proceedings of the National Academy of Sciences of the United States of America 96:10391-10396; Sherritt et al., 2003, Transplantation 75:1556-1560; Haque et al., 1998, Journal of Immunology 160:6204-6209; and Comoli et al., 2002, Blood 99:2592-2598). Furthermore, autologous EBV-CTLs are difficult to generate if 1) the SOT recipient is seronegative pretransplant, or 2) has received Rituximab. For both HCT and SOT recipients, the logistics and culture time required to generate a sufficient quantity of EBV-CTLs from a specific donor in time to treat these rapidly progressive lymphomas have been prohibitive, thus limiting their broad application.

To provide rapid access to treatment with EBV specific T-cells, the O'Reilly group and others have explored the use of HLA partially-matched off-the-shelf EBV-CTLs derived from a healthy donor other than the transplant donor (i.e. a third party donor). In 2002, Haque et al (Hague et al., 2002, Lancet 360:436-442) first reported the use of such cells in the treatment of 8 SOT recipients with EBV-PTLD including one patient with lymphoblastic lymphoma who achieved a PR. They then (Hague et al., 2007, Blood 110:1123-1131) reported a multicenter study which used this approach to treat LPD in 31 SOT and 2 HCT recipients, of whom 14 (including the 2 HCT patients) achieved CR and 3 PR. Subsequently, MSKCC reported 5 patients who developed EBV lymphoma following allogeneic cord blood or T-cell depleted HCT grafts and were treated with HLA partially matched 3rd party EBV-CTLs selected on the basis of restriction by an HLA allele shared by the alloHCT donor and/or the patient's disease. Of the 5 patients, 4 achieved a durable CR and 1 died of continued disease progression (Doubrovina et al., 2012, Blood 119:2644-2656; and Barker et al., 2010, Blood 116:5045-5049). Subsequently, limited case series have been reported, employing 3rd party off-the-shelf EBV-specific or multi-virus-specific T-cells for treatment of EBV-LPD or EBV viremia complicating cord blood or marrow HCTs, SOTs or genetic immune deficiencies (Hague et al., 2007, Blood 110:1123-1131; Barker et al., 2010, Blood 116:5045-5049; Naik et al., 2016, The Journal of Allergy and Clinical Immunology 137:1498-1505.e1; Griffith et al., 2008, The Journal of Allergy and Clinical Immunology 122:1087-1096; Leen et al., 2013, Blood 121:5113-5123; Sun et al., 2002, British Journal of Haematology 118:799-808; Uhlin et al., 2012, Clinical Infectious Diseases 55:1064-1073; Gallot et al., 2014, Journal of Immunotherapy 37:170-179; and Vickers et al., 2014, British Journal of Haematology 167:402-410). The results presented in these initial reports are summarized Table 1. However, due to the small number of patients treated in each study, analyses correlating response with characteristics of the patient, their EBV-LPD or the T-cells used for treatment have been limited.

In the following tables, CR means complete remission, PR means partial remission, SD means stable disease, POD means progression of disease, NE means not evaluated, REL means relapse, and LN means lymph nodes.

TABLE 1
Summary of reported experience with adoptive therapy with 3rd party donor
derived EBV-CTLs.

| | | | | Prior Therapy | | HLA | | | | | |
|---|---|---|---|---|---|---|---|---|---|---|---|
| Center | Method of Selection | Indication for CTLs | | Failed | N | Match | CR | PR | SD | POD | NE |
| Edinburgh | EBVBLCL Sensitized | EBV Polymorph | HCT | RIS | 2 | 2-5/6 | 2 | 0 | 0 | 0 | |
| | | Lymphoma | | | | | | | | | |
| Hague et al^{A} | EBV-CTL | EBV PTLD | SOT | Rituximab | 31 | 2-5/6 | 10 | 9 | 0 | 12 | |
| Alabama | EBVBLCL Sensitized | EBV PTLD | SOT | RT | 1 | 4/6 | 1 | 0 | 0 | 0 | |
| Sun et al^{B} | EBV-CTL | Brain | | Rituximab | 1 | 6/6 | 1 | 0 | 0 | 0 | |
| Karolinska | EBV Pentamer | EBV Lymphoma | HCT | None | 1 | 5/10 | 1 | 0 | 0 | 0 | |
| Uhlin et al^{C} | Sorted T-Cells | | | | | | | | | | |
| MSKCC | EBVBLCL Sensitized | EBV Lymphoma | HCT | Rituximab +/−C | 5 | >2/10 | 4 | 0 | 0 | 1 | |
| Barker et al^{D} | T-Cell Line | | | | | | | | | | |
| Baylor | Transduced Multivirus/incl | 8 EBV PTLD | HCT | Rituximab | 8 | ≥1 | 3 | 3 | 0 | 2 | |
| Leen et al^{E} | EBV-CTL | 1 EBV Virema | HCT | Rituximab | 1 | 1 HLA | 0 | 0 | 0 | 1 | |
| Inserm | EBVBLCL Sensitized | | HCT | Rituximab +/− C | 6 | ≥2 | 2 | 1 | 0 | 2 | 1 |
| Gallot et al^{F} | EBV-CTL | | SOT | C +/− Rituximab | 3 | ≥2 | 1 | 0 | 0 | 2 | 0 |
| Multi-Center | EBVBLCL or Multi-virus | EBVPTLD | HCT | None or | 5 | ≥3 | 1 | 1 | 0 | 3 | |
| Naik et al^{G} | Sensitized EBV-CTLs | | Immunodeficiency | Rituximab | | | | | | | |
| Aberdeen | EBVBLCL Stimulated | EBVPTLD | HCT | N/A | 6 | ≥3 | 4 | 0 | 0 | 2 | |
| Vickers et al^{H} | EBV-CTL | | SOT | N/A | 4 | ≥3 | 4 | 0 | 0 | 0 | |

^{A}Hague et al., 1998, Journal of Immunology 160:6204-6209; Hague et al., 2002, Lancet 360:436-442; and Hague et al., 2007, Blood 110:1123-1131
^{B}Sun et al., 2002, British Journal of Haematology 118:799-808
^{C}Uhlin et al., 2012, Clinical Infectious Diseases 55:1064-1073
^{D}Barker et al., 2010, Blood 116:5045-5049
^{E}Leen et al., 2013, Blood 121:5113-5123
^{F}Gallot et al., 2014, Journal of Immunotherapy 37:170-179
^{G}Naik et al., 2016, The Journal of Allergy and Clinical Immunology 137:1498-1505.e1
^{H}Vickers et al., 2014, British Journal of Haematology 167:402-410

The present study reports a single-center experience in a cohort of 46 patients with rituximab-refractory lymphomas developing post HCT or SOT who were treated with extensively characterized EBV-CTL lines between October, 2005 and January, 2015. Attributes of the disease, its prior treatment, and the T cells used for adoptive therapy that are associated with tumor response or continued progression of disease were also analyzed.

6.3. Results

6.3.1. Patient Characteristics

The characteristics of the 46 patients and their clinical and radiologic manifestations of EBV disease prior to treatment with EBV-CTLs are described in Tables 2 and 3.

TABLE 2
Demographics and pre-treatment characteristics of HCT recipients.

| | | ^{A}Time | | | | | | | | Resp- | | | | | | |
|---|---|---|---|---|---|---|---|---|---|---|---|---|---|---|---|---|
| | HCT | to | | | | | | | Doses | onse | | Resp- | | | Other | Response |
| Protocol | (HLA- | CTLs | | | | | ≥3 | # | of | to | Prior | onse | Prior | Chemo, | EBV | to Other |
| (UPN) | match) | (days) | Age | ⁸ExN | CNS | LN^{C} | sites | Rx | Ritux | Ritux | RT | to RT | Chemo | Specify | Therapy | Therapy |
| 95-024 | CBU | 34 | 8.0 | Y | N | A/B | Y | 2 | 7 | POD | N | n/a | Y | Methylpred | N | n/a |
| (5630) | (5/6) | | | | | | | | | | | | | Cyclophos | | |
| 95-024 | PBSCT | 68 | 17.4 | Y | Y | A/B | Y | 3 | 6 | PR→ | N | n/a | Y | ANHL0221 | tonsillec- | local |
| (5634) | (9/10) | | | | | | | | | POD^{D} | | | | | tomy | control |
| 11-130 | cBMT | 14 | 19.0 | Y | N | A/B | Y | 2 | 4 | SD | N | n/a | N | n/a | tonsillec- | local |
| (5627) | (8/10) | | | | | | | | | | | | | | tomy | control |
| 11-130 | TCD | 37 | 65.5 | N | N | A/B | Y | 1 | 4 | PR | N | n/a | N | n/a | N | n/a |
| (4234) | PBSCT | | | | | | | | | | | | | | | |
| | (10/10) | | | | | | | | | | | | | | | |
| 11-130 | TCD | 40 | 52.0 | Y | N | A/B | Y | 2 | 4 | POD | N | n/a | N | n/a | tonsillec- | local |
| (4835) | PBSCT | | | | | | | | | | | | | | tomy | control |
| | (9/10) | | | | | | | | | | | | | | | |
| 11-130 | TCD | 32 | 63.4 | Y | N | N | N | 1 | 4 | PR | N | n/a | N | n/a | N | n/a |
| (4513) | PBSCT | | | | | | | | | | | | | | | |
| | (10/10) | | | | | | | | | | | | | | | |
| 11-130 | TCD | 13 | 11.0 | N | N | A/B | N | 1 | 5 | SD | N | n/a | N | n/a | N | n/a |
| (5605) | BMT | | | | | | | | | | | | | | | |
| | (9/10) | | | | | | | | | | | | | | | |
| 11-130 | DUBCT | 71 | 68.4 | N | N | B | N | 2 | 8 | CR→ | N | n/a | Y | Brentux- | N | n/a |
| (5613) | (5/6; 4/6) | | | | | | | | | REL^{D} | | | | imab | | |
| 11-130 | TCD | 54 | 74.1 | Y | N | A | Y | 1 | 5 | POD | N | n/a | N | n/a | N | n/a |
| (5601) | PBSCT | | | | | | | | | | | | | | | |
| | (10/10) | | | | | | | | | | | | | | | |
| 11-130 | TCD | 17 | 23.7 | N | N | A | N | 2 | 4 | PR | N | n/a | N | n/a | tonsillec- | PR |
| (5431) | PBSCT | | | | | | | | | | | | | | tomy | |
| | (8/10) | | | | | | | | | | | | | | | |
| 11-130 | cBMT | 39 | 28.4 | Y | N | A/B | Y | 1 | 6 | PR→ | N | n/a | N | n/a | N | n/a |
| (5625) | (8/10) | | | | | | | | | POD | | | | | | |
| 11-130 | TCD | 8 | 19.7 | N | N | A/B | N | 1 | 4 | POD | N | n/a | N | n/a | N | n/a |
| (5619) | PBSCT | | | | | | | | | | | | | | | |
| | (5/10) | | | | | | | | | | | | | | | |
| 95-024 | CBU | 11 | 11.4 | Y | N | A/B | Y | 2 | 5 | POD | N | n/a | Y | HU | N | n/a |
| (5632) | (4/6) | | | | | | | | | | | | | | | |
| 95-024 | PBSCT | 37 | 63.1 | Y | N | A/B | Y | 1 | 6 | POD | N | n/a | N | n/a | N | n/a |
| (5633) | (10/10) | | | | | | | | | | | | | | | |
| 11-130 | cBMT | 8 | 28.2 | Y | N | A/B | Y | 1 | 2 | SD | N | n/a | N | n/a | N | n/a |
| (5558) | (10/10) | | | | | | | | | | | | | | | |
| 11-130 | cBMT | 43 | 19.4 | Y | N | N | Y | 1 | 5 | POD | N | n/a | N | n/a | N | n/a |
| (5626) | (10/10) | | | | | | | | | | | | | | | |
| 95-024 | CBU | 169 | 7.7 | Y | N | N | N | 2 | 14 | PR→ | Y | local | N | n/a | N | n/a |
| (5628) | (5/6) | | | | | | | | | POD | | control | | | | |
| 95-024 | TCD | 6 | 62.4 | Y | N | A/B | Y | 1 | 1 | N/A | N | n/a | N | n/a | N | n/a |
| (3520) | PBSCT | | | | | | | | | | | | | | | |
| | (10/10) | | | | | | | | | | | | | | | |
| 11-130 | DUBCT | 14 | 21.5 | Y | N | A/B | Y | 1 | 2 | POD | N | n/a | N | n/a | N | n/a |
| (5597) | (4/6; 4/6) | | | | | | | | | | | | | | | |

TABLE 3
Demographics and pre-treatment characteristics of SOT recipients.

| | | | | Time to | | | | | | | |
|---|---|---|---|---|---|---|---|---|---|---|---|
| | | | | CTLs | | | | | | | |
| | | | | from | | | | | | | |
| | | | | Most | | | ^{B}LN | | | | DISEASE |
| | Age | | ^{A}Prior | Recent | | | Above/ | | | | STATUS |
| | at | | Organ | LPD | Extranodal | | Below | ≥3 | PRIOR | PRIOR | AT START |
| UPN | SOT | Organ | Rejection | (days) | Disease | CNS | diaphragm | SITES | RT | CHEMO | OF CELLS |
| 5618 | 2.5 | HEART | Y | 34 | Y | Y | a/b | Y | N | Y | Recurrent |
| 5639 | 2.7 | SMALL | Y | 221 | Y | N | a/b | Y | N | Y | Recurrent |
| | | BOWEL | | | | | | | | | |
| 5598 | 2.0 | RENAL | Y | 448 | N | Y | N | N | N | Y | Residual |
| 5624 | 1.6 | HEART | Y | 99 | Y | N | a/b | Y | N | Y | Recurrent |
| 5637 | 3.4 | RENAL | N | 184 | Y | N | a/b | Y | Y | Y | Recurrent |
| 5606 | 24.7 | RENAL | N | 36 | N | Y | N | N | Y | N | Residual |
| 5744 | 34.9 | LUNG | Y | 142 | Y | N | a/b | Y | N | Y | Recurrent |
| 5599 | 10.7 | LIVER | Y | 160 | N | Y | N | N | Y | Y | Residual |
| 5604 | 18.1 | RENAL | N | 382 | Y | N | b | N | Y | Y | POD |
| 5620 | 75.3 | RENAL | N | 83 | N | Y | N | N | N | N | POD |
| 5640 | 0.7 | HEART | Y | 21 | Y | N | a/b | Y | N | Y | Recurrent |
| 5638 | 42.2 | LIVER | Y | 224 | N | N | a | N | N | Y | Recurrent |
| 5595 | 18.0 | HEART/ | N | 232 | N | Y | N | N | Y | Y | POD |
| | | LIVER | | | | | | | | | |

^{A}Y = Yes N = No
^{B}A = Involved Lymph Nodes above the diaphragm, B = involved lymph Nodes below the diaphragm

As summarized in Table 4, the median age at time of treatment was 23.7 years for the HCT group and 19.1 years for the SOT group. The EBV malignancy emerged at a median of 90 days (28-1545) after HCT and 1106 days (194-5330 days) after SOT. Each of these patients had been previously treated with Rituximab and had either progressed during treatment, failed to fully respond to Rituximab, or had recurred after a prior response. As detailed in Tables 2 and 3 and summarized in Table 4, 7 of 33 HCT patients and 12 of 13 SOT patients had also received chemotherapy and/or radiation therapy prior to referral for treatment with EBV-CTLs. One of the HCT patients had also received EBV-CTLs from his transplant donor and failed to respond. The median time from diagnosis of proximate episode of EBV-PTLD to treatment with EBV-CTLs was 34 days (6-169 days) for recipients of HCT, and 160 days (21-448) for recipients of SOT, the latter reflecting the duration of treatment with Rituximab+/− chemoradiotherapy administered to SOT patients prior to referral for T-cells.

TABLE 4
Summary of Demographics, extent of disease, time
to diagnosis and preceding GvHD or rejection.

| | HSCT (n = 33) | SOT (n = 13) |
|---|---|---|
| Age | 23.7 | 19.1 |
| Gender (M/F) | 15/18 | 6/7 |
| Time from Transplant to | 90 (28-1545) | 1106 (194-5330) |
| Initial Diagnosis | | |
| Time from Most Recent PTLD | 34 (6-169) | 160 (21-448) |
| Diagnosis to CTL therapy | | |
| ≥3 sites | 20 | 6 |
| 1-2 sites w/Extranodal | 7/13 | 6/7 |
| CNS | 5 | 6 |
| Extra-nodal | 25 | 7 |
| Prior GvhD or Rejection | 19/33 | 8/13 |
| Systemic Steroids | 14/22 | 5/13 |

At the time of referral for treatment with EBV-CTLs, 20/33 (59%) HCT recipients had >3/7 anatomical sites of EBV lymphoma. Of the 13 HCT patients with only 1-2 sites of involvement, 7 had disease in extranodal sites including the brain (N=2), lung (N=2), intestines (N=4), or bone (N=1). The other 6 had nodal disease only. Of the 13 SOT recipients, 6 had >3/7 sites of disease. Of the 7 with 1 or 2 involved sites, one had nodal disease only and 6 had disease in extranodal sites of brain (N=5) and bone (N=1).

6.3.2. Pathologic characteristics of EBV malignancies

The histopathologic and genetic features of the EBV-associated lymphomas are described in Table 5. As can be seen, the lymphomas were all of B cell type; and those reviewed at MSKCC were of a monomorphic diffuse large B cells lymphoma (DLBCL) histology in 24/30 HCT recipients (80%) and 8/13 SOT recipients (62%) respectively.

TABLE 5
Histology, clonality, origin and proliferative index of the post-transplant
EBV + lymphomas.

| | | | | | | Proliferative |
|---|---|---|---|---|---|---|
| UPN | Protocol | Transplant | Pathology | clonality | Origin | Index |
| 3399 | 95-024 | HCT | monomorphic DLBCL | clonal | host | 50-60% |
| | | | | | | (MIB-1) |
| 3520 | 95-024 | HCT | lymphoid hyperplasia | unknown | unknown | unknown |
| 3603 | 95-024 | HCT | monomorphic DLBCL | clonal | host | 80% |
| 4032 | 11-130 | HCT | monomorphic DLBCL | unknown | unknown | 80-90% |
| 4234 | 11-130 | HCT | monomorphic DLBCL | unknown | unknown | 90% |
| 4286 | 11-130 | HCT | monomorphic DLBCL | clonal | donor | 90% |
| 4513 | 11-130 | HCT | monomorphic DLBCL | unknown | unknown | 80% |
| 4835 | 11-130 | HCT | monomorphic DLBCL | unknown | unknown | 70% |
| 5431 | 11-130 | HCT | Mononucleosis like | oligoclonal | unknown | 5-70% |
| | | | PTLD | | | (overall 40%) |
| 5592 | 11-130 | HCT | monomorphic DLBCL | unknown | donor | >90% |
| 5593 | 11-130 | HCT | monomorphic DLBCL | clonal | host | 90% |
| | | | w/plasmacytoid | | | |
| 5597 | 11-130 | HCT | monomorphic DLBCL | clonal | donor | 90% |
| 5601 | 11-130 | HCT | Polymorphic | clonal | donor | 80% |
| 5602 | 11-130 | HCT | monomorphic DLBCL | unknown | unknown | unknown |
| 5603 | 11-130 | HCT | monomorphic DLBCL | clonal | donor | 70% |
| 5605 | 11-130 | HCT | monomorphic DLBCL | unknown | donor | 90% |
| 5609 | 11-130 | HCT | monomorphic DLBCL | clonal | donor | GI: 70-80% |
| 5613 | 11-130 | HCT | Hodgkin Like | unknown | unknown | MIB and Ki-67 |
| | | | | | | “High” |
| 5616 | 11-130 | HCT | polymorphic | not clonal | unknown | 90% |
| | | | w/plasmacytoid | | | |
| 5622 | 11-130 | HCT | monomorphic DLBCL | not clonal | donor | 90% |
| 5624 | 95-024 | HCT | monomorphic DLBCL | clonal | donor | 60-70% |
| | | | | | | (MIB-1/Ki-67) |
| 5625 | 11-130 | HCT | monomorphic DLBCL | clonal | Donor | >90% |
| 5627 | 11-130 | HCT | monomorphic DLBCL | unknown | unknown | 60-70% |
| 5628 | 95-024 | HCT | monomorphic DLBCL | clonal | Donor | >90% |
| 5629 | 95-024 | HCT | monomorphic DLBCL | clonal | donor | >90% |
| 5630 | 95-024 | HCT | monomorphic DLBCL | clonal | donor | >80% |
| | | | | | | (MIB-1) |
| 5631 | 95-024 | HCT | monomorphic DLBCL | unknown | unknown | unknown |
| 5632 | 95-024 | HCT | monomorphic DLBCL | clonal | Host | unknown |
| 5633 | 95-024 | HCT | Plasmacytoid | clonal | Unknown | unknown |
| 5634 | 95-024 | HCT | monomorphic DLBCL | unknown | donor | >90% |
| 5598 | 11-130 | SOT | mixed w/ Hodgkin-like | clonal | host | 50-60% |
| 5599 | 11-130 | SOT | monomorphic DLBCL | not clonal | host | unknown |
| 5604 | 11-130 | SOT | monomorphic DLBCL | unknown | unknown | 80% |
| | | | w/plasmacytoid | | | |
| 5606 | 11-130 | SOT | monomorphic DLBCL | unknown | recipient | 30-50% |
| 5618 | 11-130 | SOT | monomorphic DLBCL | unknown | unknown | >50% |
| 5624 | 11-130 | SOT | monomorphic DLBCL | unknown | unknown | 70% |
| 5637 | 95-024 | SOT | polymorphic DLBCL | not clonal | host | >90% |
| 5639 | 95-024 | SOT | polymorphic DLBCL | clonal | host | 80% |
| | | | | | | (MIB-1) |

In the HCT recipients the malignancy was monoclonal in 18 of 21 patients adequately tested and of transplant donor origin in 15 of 18 patients including 1 derived from a non-engrafting cord blood following a double CBT and 1 including lymphoma cells from both cord blood units following a double CBT. By contrast, in SOT recipients, 7 of 7 lymphomas tested were of host origin.

6.3.3. Characterization of EBV-CTLs Infused

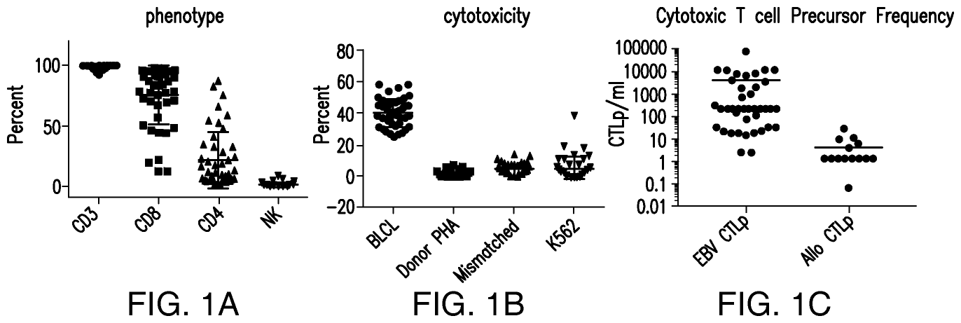

The EBV-CTLs generated in vitro were >95% CD3⁺ T cells and <1% CD19⁺ B cells (FIG. 1). The majority of EBV-CTL lines contained more than 90% CD8⁺ T cells. However, 7 CTL lines contained a majority of CD4⁺ T cells (>50% of the cell population). All T-cell lines, including those predominantly containing CD4⁺ T cells, demonstrated EBV-specific cytotoxic activity against autologous EBV-BLCLs and did not kill NK cell-sensitive targets (K562), EBV-negative autologous or recipient-derived PHA blasts, or HLA-mismatched EBV-BLCLs. In limiting dilution assays, the EBV-CTLs contained a median of 6,323.5 EBV-cytotoxic T-cell precursors (CTLps)/10⁶ cells; (range, 2.5-76,982 EBV-CTLps/10⁶ cells), and in response to an irradiated fully allogeneic PBMC, generated low or undetectable alloreactive CTLps (median 1.2 range 0-27.4 allo CTLps/10⁶ cells). EBV-CTLs administered to HCT patients did not differ significantly from those administered to SOT patients, either in the types of T-cells administered or their frequencies of EBV-CTLps (Data not shown).

The 33 alloHCT and 13 SOT recipients received a total of 103 cycles of EBV-CTLs (median=2 cycles/pt) from 55 EBV-CTL lines. Of the 55 lines, 5 were used to treat more than one patient.

HLA restrictions were identified for each of the 55 EBV-CTL lines employed: 19 (34%) were restricted by a single HLA A (N=15), HLA B (N=3) or HLA DR (N=1) allele; 36 lines (66%) were restricted by 2 (N=13), 3 (N=13), or >4 (N=10) HLA alleles.

6.3.4. Treatment with 3rd Party Derived EBV-CTL is Well Tolerated

There were no immediate adverse reactions observed due to infusion of EBV-CTLs.

One patient developed de novo grade I acute GvHD of the skin which resolved with topical therapy; none of the 19 patients with documented prior GvHD had a flare after receiving EBV-CTL therapy. After infusions of the EBV-CTLs no patient experienced de novo suppression of leukocyte, red cell or platelet counts or, in SOT patients, evidence of organ rejection.

6.3.5. Clinical Responses of EBV Associated Lymphomas to Third Party EBV-CTL Infusions

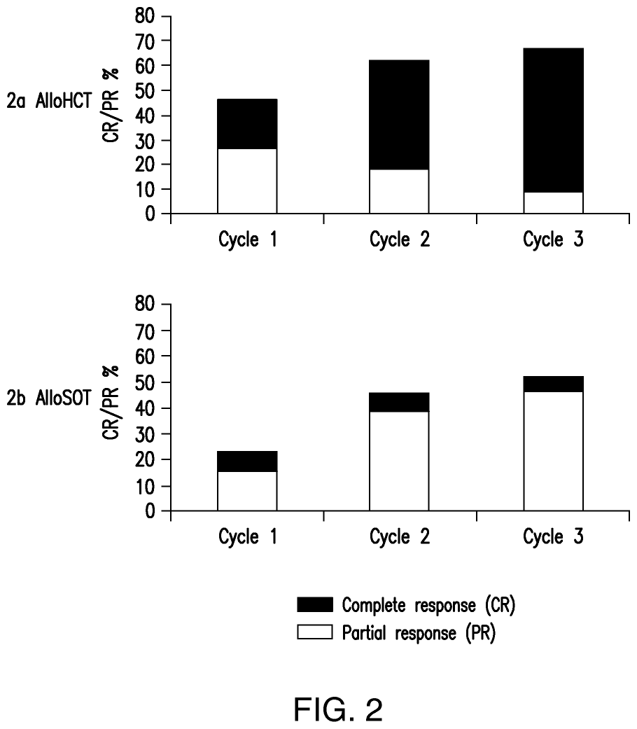

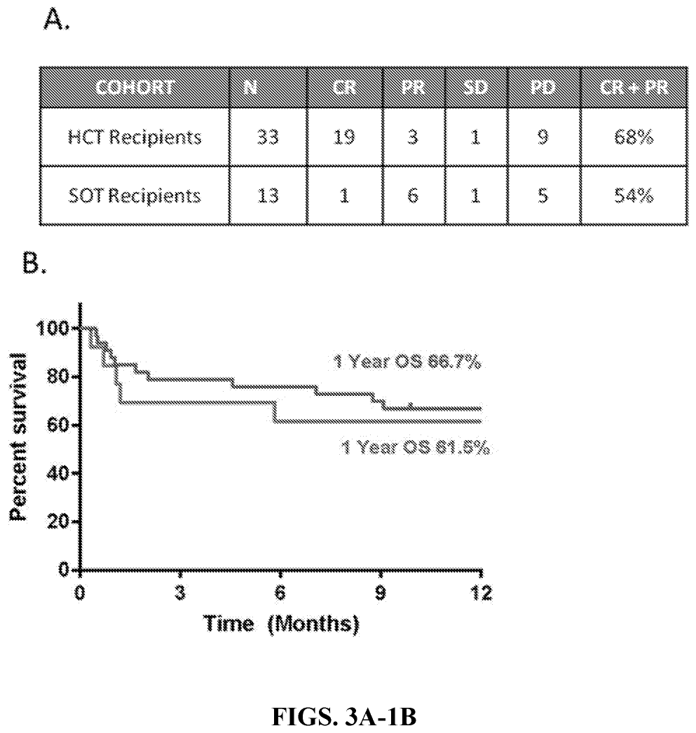

Responses to treatment with EBV-CTLs were classified as complete remission (CR), partial remission (PR), stable disease (SD), or progressive disease (PD) using the International Workshop Criteria for assessing response to treatment in non-Hodgkin lymphoma (Cheson, 2015, Chinese Clinical Oncology 4:5). Only 8/33 HCT patients and 1/13 SOT patients achieved a CR after the first cycle of EBV-CTLs. An additional 9 patients (7 HCT; 2 SOT) achieved a PR, and 10 had stable disease. Thus, the overall response (CR+PR) after cycle 1 was 39% (18/46). However, as shown in FIG. 2, response rates (CR+PR) increased with additional cycles. Maximal responses in individual patients were observed after a median of 2 cycles (range 1-5). As shown in FIG. 3A, of 33 HCT patients, 19 ultimately achieved a CR and 3 a stable PR, (CR+PR=68%). Of 13 SOT patients 1 achieved a CR but 6 achieved durable stable PRs (CR+PR=54%). In all, 29 of the 45 evaluable patients (64%) achieved a CR or sustained PR.

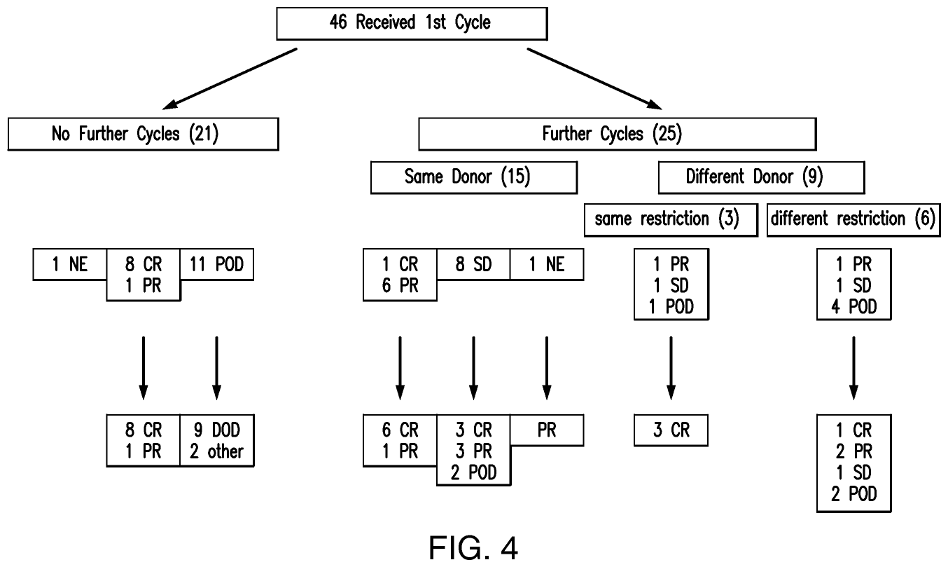

The treatment outcomes for patients treated with ≥1 cycle of EBV-CTLs are summarized in FIG. 4. Twenty-one patients received a single cycle of EBV-CTLs, of whom 8 (7 HCT, 1 SOT) achieved a CR and 1 a PR after the first cycle; the latter patient has been healthy for 4½ years, but a subsequent PET scan that was requested to assess for CR was not obtained. Eleven patients continued to have POD through the first cycle and died. Nine patients died of EBV associated lymphoma; their median survival was 28 days (10-62 days) from initiation of EBV-CTLs, reflecting its aggressiveness. One patient with POD died of sepsis during the evaluation period and one patient received an alternate therapy and responded but died 12.1 months later of GvHD that pre-dated EBV cell therapy. One other patient in the series relapsed with his primary leukemia one day after receiving his third dose of EBV-CTLs. This patient was evaluated for toxicity and is included in the 33 HCT recipients in assessments of overall survival, but could not be evaluated for EBV lymphoma response because of the chemotherapy introduced to treat the leukemia. This patient achieved remission of both EBV lymphoma and leukemia and was surviving in remission one year post treatment.

Of the 25 patients that received more than one cycle of treatment, 16 received EBV-CTLs from the same EBV-CTL cell line; 15/16 based on initial evidence of response (CR, PR or SD). Of these 15, one who had already achieved a CR remained in CR. Of 6 with a PR after the first cycle, 5 achieved a CR and one remained in PR. Of 8 patients with SD, 3 achieved a CR and 3 a durable PR. One additional patient who was not evaluable for response after a first cycle of cells due to concomitant radiation therapy received secondary cycles of cells from the same EBV-CTL cell line and achieved a PR.

Three patients, including one who achieved PR, one with SD and one with POD after cycle 1, received subsequent cycles of EBVCTLs from a different donor, but restricted by the same shared HLA allele as the primary cycle of cells. These three patients all achieved a CR.

Six patients (4 HCT with POD, 1 HCT with PR and 1 SOT with SD) received switch therapy with secondary cycles of EBV-CTLs restricted by a different HLA allele shared by the patient and, for HCT recipients, the patient's HCT donor. Based on previous analyses of EBV associated lymphoma cells isolated from patients that were not lysed by transplant donor-derived EBV-CTLs (Doubrovina et al., 2012, Blood 119:2644-2656; and Gottschalk et al., 2001, Blood 97:835-843) it was reasoned that switching to EBV-CTLs specific for a different epitope presented by an alternate shared HLA allele might better treat an EBV lymphoma that was initially resistant. Of these 6 patients, one with POD after the first cycle ultimately achieved a CR; a second with POD after the first cycle achieved a durable PR. The patient with PR after the first cycle has remained in PR; the patient with SD also remained with SD. The other 2 patients in POD continued with POD. However, progression was slowed in comparison to others with POD after cycle 1 with survival extended to 215 and 266 days respectively.

Among those patients who achieved a CR or PR, clinical improvements, including shrinkage of palpable lymph nodes, reduction of organomegaly, and resolution of pain or intestinal bleeding, were first detected 8-15 days after infusion of the effective T cells. Similarly, improvements in radiologic/endoscopic findings were documented by 28-35 days after the start of therapy. In patients with stable disease, symptoms including pain and fever plateaued or improved, but radiographic abnormalities were unchanged. In contrast, patients who failed to respond showed persistence of fever and other clinical symptoms with continued clinical deterioration and/or worsening of radiologic findings.

In responding patients who had detectable EBV DNA levels in the blood prior to T-cell infusion, EBV DNA levels fell by 2 log₁₀ post infusion, and were a useful indication of response. However, because these patients had been previously treated with rituximab, EBV DNA was often not detectable in the blood prior to or after T-cell infusions. Thus, in 13 HCT patients and 6 SOT patients EBV DNA was not detected in the blood even though these patients at the same time, had clinical and/or radiologic evidence of POD.

Median follow-up for HCT recipients is 12.6 months and 16.8 months for SOT recipients. Both the complete, and, strikingly, the partial remissions, in both the HCT and SOT groups have been durable (>6->115 months). The overall survival (OS) at 1 year is 66.7% for HCT and 61.5% for SOT recipients (FIG. 3B). The cumulative incidences of EBV-specific mortality at 12 months were 27% (±8%) for recipients of HCT, and 38% (±14%) for recipients of SOT. All deaths attributable to EBV-LPD occurred within 8.8 months of initiation of T-cell therapy in HCT and 5.8 months for SOT recipients.

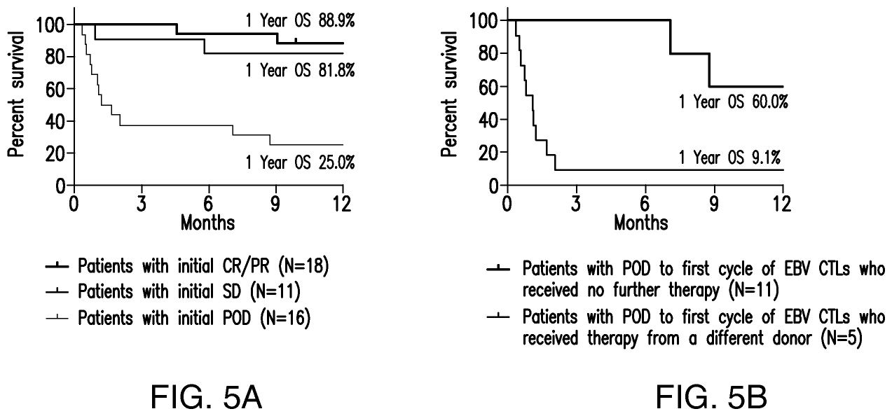

FIG. 5A presents a K-M plot of overall survival for the 29 patients who achieved a CR, PR or stable disease following their first cycle. Survival for patients achieving CR or PR is 88.9% at 1 year, and 81.8% for those who had stable disease. For those with POD after cycle 1, overall median survival was 44 days; 1 year survival is 25%. As shown in FIG. 5B, only 1/11 with POD (9.1%) who received only 1 cycle survived a year. In contrast, for those who received secondary cycles of EBV-CTLs from a different donor, survival at 1 year is 60%.

6.3.6. Clinical and Immunologic Variables Affecting Outcome

6.3.6.1. Clinical Characteristics of Patients Associated with Response

The patient characteristics examined for an association with response are summarized in Table 6. All sites of involvement with PTLD, including the CNS, responded to EBV-CTL therapy. Indeed, of the 11 patients with clinical and radiologic evidence of CNS involvement, 3 achieved CR and 6 durable PRs. The proportion of HCT and SOT recipients with multiple sites of disease who achieved a CR or sustained PR (52%) was lower than that observed in patients with <3 sites of disease (80%) but did not reach statistical significance (P=0.07). Overall, patients with extranodal sites of lymphoma had a lower response rate (p<0.001). Patients treated with Rituximab alone prior to EBV-CTLs fared better than those previously treated with both Rituximab and chemotherapy (80% vs 45%, p=0.03).

TABLE 6
Comparison of clinical and treatment variables predicting response to 3^{rd}
party EBV-CTL therapy.

| | Overall | | HSCT | | | |
|---|---|---|---|---|---|---|
| | Responder | | Responder | | SOT | |
| | (%) | | (%) | | Responder (%) | |
| | Responder | | Responder | | Responder | |
| | (%) | p value | (%) | p value | (%) | p value |
| Rituximab Only | 20/25 (80%) | 0.03 | 19/24 (79%) | 0.07 | 1/1 (100%) | 0.47 |
| Rituximab + Other | 9/20 (45%) | | 3/8 (38%) | | 6/12 (50%) | |
| Age ≥ 50 years | 10/15 (67%) | 0.99 | 8/13 (62%) | 0.7 | 2/2 (100%) | 0.46 |
| Age < 50 years | 19/30 (63%) | | 14/19 (74%) | | 5/11 (45%) | |
| Sits of Disease | | | | | | |
| ≥3 sites | 13/25 (52%) | 0.067 | 12/19 (63%) | 0.47 | 1/6 (17%) | 0.03 |
| <3 sites | 16/20 (80%) | | 10/13 (77%) | | 6/7 (86%) | |
| CNS | 9/11 (82%) | 0.28 | 4/5 (80%) | 0.99 | 5/6 (83%) | 0.1 |
| No CNS | 20/34 (59%) | | 18/27 (67%) | | 2/7 (29%) | |
| Extranodal | 16/31 (52%) | <0.01 | 15/24 (62%) | 0.38 | 1/7 (14%) | <0.01 |
| | | (0.008) | | | | (0.005) |
| No extranodal | 13/14 (93%) | | 7/8 (88%) | | 6/6 (100%) | |
| Prior GvHD or | 15/26 (58%) | 0.34 | 11/18 (61%) | 0.26 | 4/8 (50%) | 0.99 |
| rejection | | | | | | |
| No prior GvHD or | 14/19 (74%) | | 11/14 (79%) | | 3/5 (60%) | |
| rejection | | | | | | |
| Systemic steroids | 11/19 (58%) | 0.53 | 9/14 (64%) | 0.71 | 2/5 (40%) | 0.59 |
| No systemic | 18/26 (69%) | | 13/18 (72%) | | 5/8 (62%) | |
| steroids | | | | | | |

Although both CRs and PRs have been durable in HCT and SOT recipients, the overall response rate (CR+PR) and particularly, the CR rate in HCT patients was higher than that achieved by SOT recipients (68% vs 54% (P=0.50) and 58% vs 8% (P=0.003) respectively). Since EBV-CTLs are expected to be susceptible to rejection following adoptive transfer into patients with residual T-cell function, it was examined as to whether differences in the number or function of endogenous T-cells in the HCT and SOT recipients might be correlated with the differences in response observed. As shown in Table 7, there were no significant differences in the numbers of CD4 or CD8 T-cells, or T-cell responses to phytohemagglutinin (PHA) prior to adoptive therapy between responders and non-responders in either the HCT or SOT group. However, the CD4 and CD8 levels, as well as the PHA responses in the overall HCT group were significantly lower than those of the SOT recipients (p, 0.002), reflecting their greater degree of T-cell deficiency prior to treatment.

TABLE 7
Recipient baseline immune phenotype and function.

| | HCT Cohort | | | SOT Cohort | | |
|---|---|---|---|---|---|---|
| | Responder | Non-Responder | | Responder | Non-Responder | |
| | Median | Median | p | Median | Median | p |
| CD3 + cells/mc | 163 | 130 | 0.11 | 835 | 339 | 0.37 |
| CD3 + 4 + cells/mc | 75 | 22 | 0.46 | 253 | 126 | 0.29 |
| CD3 + 8 + cells/mc | 103 | 103 | 0.65 | 568 | 343 | 0.53 |
| Phytohemagglutinin cpm | 5335 | 5344 | 0.39 | 59520 | 43350 | |

Although this is a limited data set, no significant differences in response were seen when EBV-CTLs were administered with or without concomitant steroid therapy. Of 19 patients receiving steroids (Table 6) at the time of cell therapy, 11 responded (58%) compared to 18/26 (69%) patients who were not receiving steroids. Of those receiving ≥0.2 mg/Kg/day prednisone or its equivalent, 3/7 (43%) responded, compared to 8/12 (67%) patients receiving <0.2 mg/Kg.

6.3.6.2. Characteristics of EBV-CTLs and In Vivo EBV-CTL Proliferation Post Transfer Associated with Response

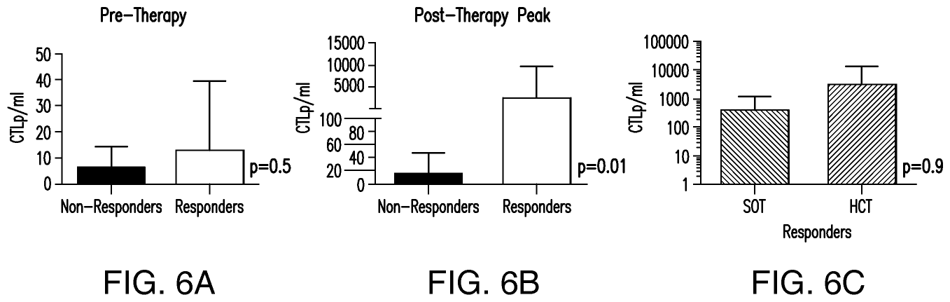

Whether treatment outcome was associated with any of several characteristics of the EBV-CTLs infused was examined. As shown in FIG. 6, lines used to treat patients who did or did not achieve a CR or PR did not differ significantly in the dose of EBV-specific CTLp/kg administered (p=0.94). The distribution of CD4+ and CD8+ T-cells in the EBV-CTLs administered was also similar. CD4 T-cells accounted for a median of 11% in the EBV-CTLs administered to patients who achieved a CR or PR, compared to 9% in patients who failed to respond (p=0.58).

Strikingly, the degree of HLA matching between the EBV-CTLs administered and the HCT donor and patient or the SOT patient was not correlated with response. In this series, the EBV-CTLs were matched with the patient and, for HCT, the transplant donor at a median of 4/10 alleles. Of 19 patients treated with EBV-CTLs matched for 1-3 HLA alleles, 12 (63%) achieved a CR or PR compared to 17/26 (65%) of patients treated with EBV-CTLs matched for 4-8 alleles (p=0.99). Response rates for EBV-CTLs matched for 1-3 vs. 4-8 HLA alleles were also similar for patient groups analyzed by transplant type. The degree of HLA matching between EBV-CTLs and the patients was also examined using the Cochran-Armitage test to identify any trends in compatibility associated with response. Again, no significant relationship was observed between the numbers of matched HLA alleles between the EBV-CTLs and the patients to whom they were given and the achievement of a response (CR or PR) (p=0.52). Although the EBV lymphomas emerging in the recipients of HCT were of transplant donor type in 15/19 cases, also no significant trend was found in response between the number of HLA alleles shared by the EBV-CTLs and the HCT donor (p=0.98).

Whether the number of HLA restrictions exhibited by the EBV-CTLs that were shared by the patient and the transplant donor (for HCT recipients) affected outcome was also examined. Overall, of 28 patients treated with EBV-CTLs restricted by a single shared HLA allele, 16 (57%) achieved a CR or PR, compared to 13/17 patients (76%) responding to EBV-CTLs restricted by more than one HLA allele shared by the patient (HCT and SOT) and donor (HCT). Although these data suggest an advantage for EBV-CTLs restricted by multiple alleles shared by the EBV associated lymphoma, in this limited series, the difference in response was not significant (p=0.22).

For those patients treated with the EBV-CTLs restricted by a single shared HLA allele, the restricting HLA alleles included 13 class I and one class II HLA alleles. In those patients treated with EBV-CTLs containing T-cells restricted by more than one HLA allele shared by the patient, 13/17 EBV-CTL lines also included T-cells restricted by at least one of this same group of HLA alleles. In this limited series, no association between administration of EBV-CTLs restricted by any specific HLA allele and clinical response was detected.

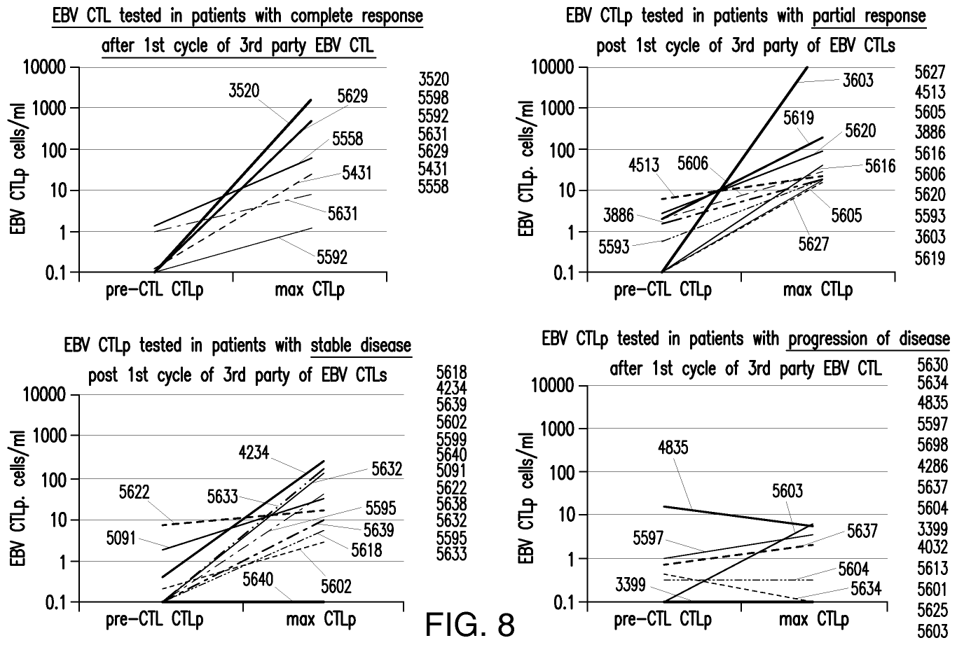

Although this study was unable to identify characteristics of the EBV-CTLs infused that predicted response, infusions of EBV-CTLs regularly resulted in increased blood levels of EBV-CTLps during the first cycle in those patients who subsequently achieved a CR or PR in response to those EBV-CTLs (FIG. 8). In contrast, with the exception of one patient who died rapidly of progressive disease, EBV-CTLp frequency expansion was not observed in patients whose disease progressed through a cycle of EBV-CTLs. Among responders, EBV-CTLp frequencies increased by a mean of 200 fold over pre-infusion levels compared to 2 fold (range 0-4) increase in non-responders (p=0.001). Increases in frequency of EBV-CTLp observed in the blood of patients responding to their first cycle were usually detected by 10-21 days after the initial infusion and coincided with clinical improvement. Increases in EBV-CTLp were also detected in 6/7 patients with SD after the first cycle who ultimately achieved a CR or PR.

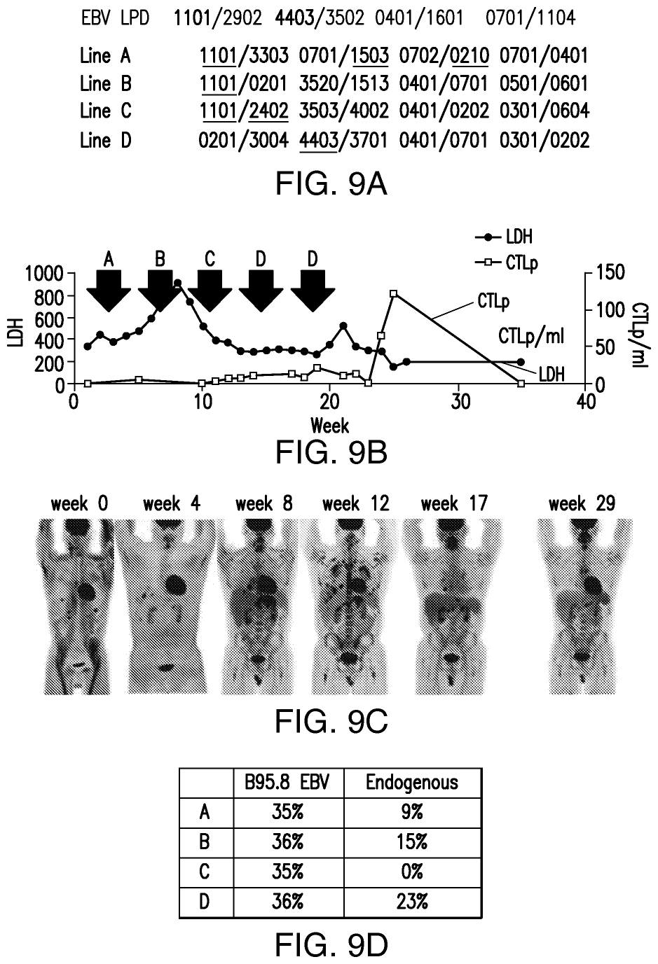

The contrast between the expansion of EBV-CTLp in patients who achieved a CR or PR and the lack thereof in patients who failed to respond was also observed in the same patient among those who failed to respond to EBV-CTLs restricted by one HLA allele, but subsequently responded to a secondary EBV-CTL line restricted by a different shared HLA allele. This is exemplified by patient UPN5997, illustrated in FIG. 9, who had developed an EBV lymphoma of cord blood transplant donor origin and initially had a mixed response with subsequent POD after treatment with 3 separate EBV-CTL lines restricted by the same shared HLA allele, HLA A*11:01. During these cycles increases in CTLp frequencies were not observed. He was then switched to an EBV-CTL line restricted by another shared allele, HLA B*44:03. This line induced a CR, associated with a marked increment in CTLp frequencies. In this care, it is important to note that 2 of the 3 EBV-CTL lines restricted by HLA A*11:01 that were infused had induced a CR or PR in other patients. However, in this case, it was found that these HLA A*11:01 restricted EBV-CTL lines that were specifically cytotoxic against autologous and HLA A*11:01+ allogeneic B cells transformed by the B95.8 strain of EBV, failed to lyse the HLA A*11:01+ cord blood-derived spontaneously transformed B-cells that were grown from the patient's lymphomatous tonsil. In contrast, the EBV-CTL line restricted by HLA B*44:03 that induced a CR was cytotoxic against both the patient's HLA B*44:03⁺ EBV associated lymphoma cells and against HLA B*44:03⁺ B cell lines transformed with the B95.8 strain of EBV. These results suggest that the patient's EBV associated lymphoma failed to express an epitope presented by HLA A*11:01 that could be recognized by the EBV B95.8 BLCL sensitized HLA A*11:01-restricted EBV-CTL lines. However, his lymphomadid express an epitope presented by HLA B*44:03 that could be recognized by an EBV-CTL line restricted by that allele.

6.4. Discussion

This study details the effects of HLA partially matched “off the shelf” 3rd party EBV-CTLs, restricted by an HLA allele shared by the patient's disease, in the treatment of 46 recipients of allogeneic HCT or SOT with EBV+ lymphomas who had failed treatment with Rituximab. The EBV-CTLs selected for each patient are derived from a bank of 330 cryopreserved EBV-CTL lines, generated under GMP conditions from the blood of healthy, specifically consented donors, each extensively pre-characterized as to microbial sterility, high resolution HLA type, immunotype, lack of alloreactivity, EBV specificity and HLA restriction. This pre-characterization permitted rapid selection of appropriately HLA restricted EBV-CTLs, and treatment within as few as 1-2 days of patient referral.

Because these EBV-CTLs are allogeneic to both transplant donor and recipient, ascertaining their safety was a principal goal. The banked EBV-CTLs were generated for 28-35 days employing only autologous EBV B95.8 transformed B cells for sensitization and expansion. This culture period yields EBV-specific T-cells that are depleted of alloreactive T-cells, as ascertained by their failure to lyse allogeneic EBV-negative targets and the minimal frequencies of CTLp generated in response to allogeneic EBV-negative stimulators (FIG. 1). In vivo, the EBV-CTLs were well tolerated. In particular, no HCT or SOT recipient exhibited evidence of graft rejection or a flare of GVHD; de novo GVHD was observed in only one patient who experienced a grade 1, transient skin rash. Furthermore, clinical responses were not associated with features of the cytokine release syndrome that has been observed in patients responding to non-specifically activated autologous or allogeneic T-cells transduced to express a chimeric antigen receptor (Lee et al., 2014, Blood 124:188-195). Thus, adoptive transfer of these EBV-CTLs has been safe.

The 3^{rd} party EBV-related lymphomas targeted in this study uniformly presented as rapidly developing B cell malignancies, that were monomorphic DLBCLs in 80% of the cases. Of the 46 patients, 26 (55%) had disease in >3 anatomic sites; of the other 20 patients, 14 (66%) had extranodal disease, including 7 with disease in the CNS. All of these patients had failed or relapsed following treatment with Rituximab. Furthermore, 9/33 HCT and 12/13 SOT recipients had failed treatment with both rituximab and chemotherapy. These disease characteristics have been associated with a uniformly poor prognosis (Choquet et al., 2007, Annals of Hematology 86:599-607). Nevertheless, 68% of the HCT recipients and 54% of the SOT recipients treated with 3^{rd} party EBV-CTLs achieved a CR or durable PR.

For HCT recipients, the response rate is similar to that which has previously been reported for patients with Rituximab-refractory EBV lymphomas treated with transplant donor-derived EBV-CTL (Doubrovina et al., 2012, Blood 119:2644-2656). However, patients treated with transplant donor-derived EBV-CTL usually achieved a complete response by 3 weeks after a single cycle of 3 EBV-CTL infusions. In contrast, only 39% of the patients treated with EBV-CTL achieved a CR or PR after cycle 1; and an additional 19% stable disease. However, with additional cycles of EBV-CTLs 6/8 patients in initial PR achieved a CR and two remained in PR. Strikingly, 7/10 patients with initial SD that received this treatment also achieved a CR (N=3) or PR (N=4). These findings illustrate that responses to the EBV-CTLs were cumulative and provide evidence that initial stabilization of disease may indicate responsive disease warranting continued treatment with EBV-CTLs.

An important objective of this study has been the identification of attributes of the 3^{rd} party EBV-CTLs that are predictive of their clinical effectiveness. Haque et al (Hague et al., 2007, Blood 110:1123-1131) reported higher response rates in patients who received EBV-CTLs that were more closely matched for HLA A, B and DR alleles and EBV-CTLs containing more than 1% CD4+ T-cells. In contrast, this study did not find a significant correlation between degree of HLA match and subsequent response. However, in this trial, selection of EBV-CTLs was primarily based on their restriction by an HLA allele that would be shared by the patient's EBV associated lymphoma. This prioritization was based on a previous trial of transplant donor-derived EBV-CTLs in which a failure to respond in one patient could be ascribed to restriction of EBV-CTLs from the HLA haplotype matched parental HCT donor that were infused by an HLA allele not shared by host-derived EBV lymphoma. This patient subsequently responded to EBV-CTLs restricted by an HLA allele shared by that lymphoma (Doubrovina et al., 2012, Blood 119:2644-2656). Since 3^{rd} party EBV-CTLs are rarely fully HLA-matched, and EBV-CTLs generated from latently infected normal donors are usually specific for a limited number of epitopes, presented by only 1-3 HLA alleles, the ability to select EBV-CTL lines based on their predetermined HLA restriction has distinct advantages. This is especially the case when treating an EBV lymphoma of undefined origin in recipients of HLA disparate HCT, SOT or cord blood grafts for which an EBV-CTL restricted by an HLA allele shared by both the transplant donor and recipient addresses both possibilities.

This study and those of Leen et al (Leen et al., 2013, Blood 121:5113-5123; and Tzannou et al., 2017, Journal of Clinical Oncology 35:3547-3557) also found no correlation between the proportion of CD4+ T-cells in the T-cells administered and subsequent response. However, Haque et al (Hague et al., 2007, Blood 110:1123-1131), were almost exclusively treating organ allograft recipients. In these demonstrably less immunosuppressed individuals, transferred CD4+ T-cells may foster short-term engraftment and a more rapid and extensive expansion of CD8+ T-cells. Larger trials will be required to ascertain whether and to what degree CD4+ EBV-CTLs enhance response rates in SOT recipients.

Prior studies (Doubrovina et al., 2012, Blood 119:2644-2656; Barker et al., 2010, Blood 116:5045-5049; Leen et al., 2013, Blood 121:5113-5123; and Rooney et al., 1998, Blood 92:1549-1555) have reported a significant correlation between the in vivo expansion of EBV-CTLs following adoptive transfer of transplant donor-derived EBV-CTLs and clinical response. In this study, increases in the frequency of EBV-specific CTLps were regularly detected 10-21 days into the first cycle in patients who achieved CR or durable PR, as well as in patients with SD who subsequently attained a CR or PR.

Multiple factors could be invoked to explain the progression of disease that occurred despite treatment with appropriately HLA-restricted EBV-CTLs. Disease status clearly plays a role, since patients with extensively pre-treated lymphomas at time of referral had a poorer prognosis. EBV also employs an array of factors by which it could evade EBV-CTL, ranging from epitope variation between different strains of EBV (Khanim et al., 1996, Blood 88:3491-3501; and Rajcani et al., 2014, Recent Patents on Anti-Infective Drug Discovery 9:62-76) to viral encoded proteins and microRNAs that can prevent antigen processing and presentation, or directly inhibit T-cell function (Ressing et al., 2008, Seminars in Cancer Biology 18:397-408). Indeed, the consistent correlation that has been observed between disease progression and failure of the adoptively transferred EBV-CTLs to expand in vivo has suggested either that the T-cells are rapidly eliminated by antibodies or residual alloreactive T-cells in the host or that the EBV-CTLs are unable to recognize the patient's EBV lymphoma. Doubrovina et al. (Doubrovina et al., 2012, Blood 119:2644-2656) previously reported three patients who failed to respond to B95.8 EBV sensitized HCT donor-derived EBV-CTLs, even though the EBV lymphomas in the patients were of HCT donor origin. In these cases, EBV-CTLs used also failed to lyse spontaneously transformed EBV+ BLCLs generated from the patient's HCT donor-type EBV associated lymphoma. In contrast, the donor T-cells, sensitized with the autologous tumor cells grown from the patients' EBV+ lymphomas, were able to lyse both endogenous virus and the B95.8 virus transformed B cells, thus suggesting that the tumor cells did not have a defect in antigen presentation, but rather that the endogenous virus presented an EBV antigen not expressed by B95.8 EBV+ BLCLs. In a similar case, Gottschalk et al (Gottschalk et al., 2001, Blood 97:835-843) demonstrated that spontaneous EBV+ transformed B cells isolated from a patient who failed to respond to B95.8 EBV-sensitized T-cells contained an endogenous EBV strain with a mutation that resulted in deletion of two epitopes of EBNA 3C presented by HLA A1101 that were selectively targeted by those T-cells.

Based on these findings, it was hypothesized that if an EBV strain lacked an epitope presented by one restricting HLA allele, selection of an alternate CTL line, particularly one specific for an epitope presented by another HLA allele shared by the patient's disease, might prove effective. This “switch” therapy has induced complete or durable partial remissions in 3 of the 5 patients who had POD after their first cycle. It was also observed that in one of these patients, EBV-CTLs restricted by HLA A*11:01 from 3 donors that had failed to expand in vivo or induce a clinical response, also failed to induce significant lysis of the patient's EBV associated lymphoma cells. In contrast, subsequent infusions of EBV-CTLs from an alternate donor that were restricted by HLA B*44:03, and could lyse the patients' EBV associated lymphoma cells in vitro, induced a complete remission of disease, and, concurrently, a rise in EBV-CTLp frequencies in the blood.

These findings thus provide evidence that sequence variations in the latent EBV proteins affecting epitopes presented by different strains of EBV may render EBV associated lymphomas emerging post transplant insensitive to EBV-CTLs sensitized with EBV BCLs transformed by the B95.8 strain of EBV. However, they also demonstrate that in patients with continued progression of disease, switching to EBV-CTLs specific for another epitope presented by a different shared HLA allele can provide effector cells able to recognize the tumor, and induce complete or durable partial remissions of disease. In particular, since 9 of the 11 patients with POD who received only 1 cycle of cells, died of lymphoma at a median of 28 days post initiation of EBV-CTL therapy, patients who continue to progress through the first 21 days of a cycle should be considered for such “switch therapy” immediately, forgoing the observation period.

A striking, but yet poorly understood, finding has been the durability of both CRs and PRs observed following adoptive transfer of EBV-CTLs. Because these T-cells are allogeneic to both the HCT donor and recipient, and lack demonstrable alloreactivity, these T-cells are expected to be rejected relatively rapidly. Indeed, the rationale for giving repeated three-week cycles of cells was based on the hypothesis that such repeated doses would provide a more sustained exposure to the adoptively transferred EBV-CTLs. The cumulative nature of the responses observed following cycles of 3^{rd} party EBV-CTLs, provides indirect evidence consistent with this hypothesis, as does the predominance of PRs observed in SOT patients which contrasts with the preponderance of CRs achieved in the more T-cell deficient HCT recipients. The origin of the EBV-CTLps that expanded in vivo in responding patients was not genetically ascertained. However, the increments in CTLp frequencies observed after each dose suggest that these cells usually persist for no more than 2-3 weeks after each cycle. This would suggest that while the initial responses are closely related to the increases in EBV-CTLp regularly detected 10-21 days post infusion and can be reasonably ascribed to their effector function, the durability of responses observed is more likely due to activation of endogenous T-cell responses potentially stimulated by cross presentation of antigens from EBV associated lymphomas destroyed by the 3^{rd} party EBV-CTLs and potentially by recruitment of endogenous T-cells responding to the allogeneic 3^{rd} party effector cells. However, it is also possible that small populations of the 3^{rd} party EBV-CTLs persist. Indeed, Leen (Leen et al., 2013, Blood 121:5113-5123) and Hague (Hague et al., 2007, Blood 110:1123-1131) documented persistence 3rd party T-cells in patients 22 to up to 94 days post infusion respectively. Ongoing studies of the distribution and fate of genetically distinguishable 3^{rd} party EBV-CTLs should clarify the relative contribution of the adoptively transferred EBV-CTLS and any endogenous T-cells subsequently generated to the enduring responses observed.

In summary, 3rd party EBV-CTLs that are partially HLA matched and appropriately HLA restricted can induce durable CRs or PRs in a high proportion of HCT and SOT patients with high risk, Rituximab refractory EBV lymphomas without significant toxicity, graft injury or GVHD. Maximal responses are cumulative, requiring on average, two 3-week cycles of EBV-CTL infusions. However, patients responding to a particular EBV-CTL line distinctively exhibit increases in the frequency of EBV-specific T-cells in the blood within 10-21 days of the first infusion. Furthermore, patients with progressive disease after 1 cycle can respond to treatment with an alternate EBV-CTL line specific for a different epitope presented by an alternate HLA allele shared by the lymphoma. Thus, off-the-shelf EBV-CTLs provide immediately accessible options for potentially curative treatment of high risk EBV lymphomas complicating HCT or SOT.

6.5. Materials and Methods

6.5.1. Experimental Design

Patients developing EBV viremia and/or EBV-PTLD were enrolled onto protocol prior to being treated with EBV-CTLs. All patients gave consent and were treated on one or the other of two consecutive protocols evaluating adoptive therapy with EBV-CTLs approved by the Institutional Review/Privacy Board at Memorial Sloan Kettering Cancer Center, the Food and Drug Administration and the National Marrow Donor Program. The first of these protocols, introduced in 1995, initially evaluated HCT-donor-derived EBV-specific T-cells, but was amended to permit treatment with partially HLA-matched, appropriately HLA-restricted T-cells from third party donors. The second was introduced in 2011, specifically to evaluate adoptive immunotherapy with 3rd party EBV-CTLs. Both protocols were single armed Phase II trials. Patients eligible included HCT or SOT recipients with pathologically confirmed EBV associated lymphomas. Treatment consisted of EBV-CTLs matched with the patient for 2/10 HLA alleles by high resolution typing (HLA-A, B, C, DR or DQ) and restricted by an HLA allele shared by the EBV lymphoma (when origin was known), the HCT donor and patient in HCT recipients or the patient in SOT recipients. A treatment cycle consisted of 3 weekly intravenous infusions of 1×10⁶/kg (on Protocol 1) or 2×10⁶/kg (on Protocol 2) EBV-CTL/kg, followed by a 3-week period of observation. Patients who failed to achieve CR and had no therapy related toxicity could receive additional cycles or be referred for an alternate therapy. Primary end points for these studies were: 1) incidence of complete or partial responses as determined by clinical and radiographic criteria, and 2) incidence of infusion related toxicities, including alterations of HCT or SOT function or GVHD. Secondary end points included: 1) alterations in EBV DNA levels and 2) alterations in EBV-CTL precursor frequency measured in sequential blood specimens obtained prior to and at defined intervals following each infusion.

6.5.2. Patients

A total of 46 patients received 3rd party EBV-CTLs between October 2005 and January 2015 as treatment for EBV associated B cell lymphomas that developed after an alloHCT (N=33) or SOT (N=13) and were refractory to or relapsed after therapy with rituximab.

6.5.3. Donors

EBV-CTLs were generated from a leukopheresisleukopheresis or unit of blood provided by healthy EBV-seropositive HCT donors who specifically consented to these donations for the expressed purpose of generating EBV-CTLs for use in adoptive immune therapy of an EBV-associated malignancy developing in either the recipient of their HCT or other patients with EBV-associated malignancies.

6.5.4. Diagnosis and Characterization of EBV-LPD

The EBV associated lymphomas were classified according to the WHO criteria (Swerdlow et al., 2016, Blood 127:2375-2390). Biopsy specimens were tested for EBV by in situ hybridization for EBER and in some cases by immunohistology for LMP-1. They were also tested for B and T cell markers. Whenever possible, the EBV+ tumor cells were examined for clonality of the B cells and their origin (host or donor). The genetic origin of the lymphoma was identified as donor or host, using FISH for XX vs XY in sex mismatched transplants, and by donor or host unique PCR-amplified short tandem repeat (STR) polymorphisms. Clonality of the tumors was identified by analysis of immunoglobulin rearrangements (Inghirami et al., 1993, Laboratory Investigation 68:746-757). Clonality of the EBV virus was determined by the method of Guilley and Raab-Traub (Gulley and Raab-Traub, 1993, Archives of Pathology & Laboratory Medicine 117:1115-1120).

6.5.5. Generation and Characterization of EBV-CTL Lines

The EBV-CTLs were derived from a bank of 330 EBV-CTL lines generated from leukocytes donated from specifically consenting HCT donors under FDA compliant, Good Manufacturing Practice (GMP) conditions as previously described (Doubrovina et al., 2012, Blood 119:2644-2656). Briefly, T cells were enriched from PBMCs by depletion of monocytes by adherence to plastic and natural killer (NK) cells by adsorption to anti-CD56 immunomagnetic beads (Miltenyi Biotec). T cells were sensitized in vitro at a 20:1 responder: stimulator ratio with irradiated autologous EBV transformed B cells (EBV-BLCLs) generated previously by transformation with the B95.8 strain of EBV (kindly provided by C. Rooney, Baylor College of Medicine). T cells were then cultured in Yssel medium (Gemini Bioproducts) supplemented with 5% heat-inactivated pooled normal human serum and re-stimulated with the same EBV-BLCLs weekly at a 4:1 responder: stimulator ratio.

Beginning on day 16, IL2 (Novartis) was added at 10-50 IU/mL 3 times/wk. After 28-35 days of culture, T cells were enumerated and characterized by flow cytometry using mAbs against CD3, CD4, CD8, CD56, CD19, TCRa/f3, CD28, and CD45RA (BD Biosciences). EBV-specific cytotoxicity, lack of alloreactivity, and HLA restrictions of the EBV-CTLs were identified by assessing their cytotoxicity against autologous donor- and patient or fully allogeneic donor-derived EBV⁺ BLCL and EBV⁻ phytohemagglutinin (PHA) blasts and thereafter against a panel of allogeneic EBV-BLCLs, each sharing a single HLA allele expressed by the T cells as previously described (Doubrovina et al., 2012, Blood 119:2644-2656).

T cells meeting release criteria for use in adoptive therapy were aliquoted into labelled vials and cryopreserved. These release criteria included: 1) microbial sterility demonstrated by negative cultures for bacteria, fungi and mycoplasma and endotoxin levels ≤5 EU/ml cell dose; 2) specific cytotoxic activity against autologous EBV transformed B-cells but not against autologous PHA blasts in standard ⁵¹CR release assays and 3) absence of significant cytotoxicity against patient or allogeneic donor PHA blasts or against fully HLA mismatched EBV BLCL in standard ⁵¹CR release assays.

The frequency of EBV-specific and alloreactive CTL precursors (CTLps) in EBV-CTL lines was measured by limiting dilution analysis (Lucas et al., 1996, Blood 87:2594-2603) using irradiated autologouls EBV-BLCLs as the stimulator cell for EBV-CTLp and irradiated, fully allogeneic PBMC as the stimulators for allogeneic-CTLp. When possible, the ability of EBV-CTLs to recognize endogenous EBV derived from a patient was assessed by measuring cytotoxic activity against spontaneously transformed EBV⁺ B cells cultured from either a tumor biopsy or the PBMCs of the patient with EBV PTLD.

6.5.6. Monitoring of Patients

All patients were monitored sequentially for response by clinical assessments; by imaging with CT, PET/CT, and/or MRI imaging prior to, and at the end of each cycle, or as clinically indicated. EBV DNA copy numbers in the blood were monitored from 1995-2003 with a semiquantitative PCR-amplified assay and since 2003 with a quantitative real-time PCR assay EBV-CTLp frequencies were quantified before adoptive transfer of the EBV-CTLs and thereafter on days 1, 7, 14, 21, and 28, and monthly for 4 months. Patients were also monitored closely for serious adverse events using standard seriousness criteria the National Cancer Institute common toxicity grading criteria and for acute GVHD as graded by the NCI consensus criteria (http://www.hrc.govt.nz/sites/CTCAEmanualDMCC.pdf), and via regular safety assessments including standard hematology and chemistry measures.

6.5.7. Statistical Methods

The Kaplan-Meier method was used to estimate the probability of survival over time. Comparisons of response rates between groups were assessed with Fisher's exact test. The Cochran-Armitage test was used to determine whether there was a trend between the degree of HLA matching between the EBV-CTL donor and either the transplant donor or recipient and the patient's response. The Wilcoxon rank sum test was used to evaluate whether the CD4 or CTLp frequencies in the EBV-CTLs administered differed as a function of response.

6.5.8. Protocols

Protocols NCT01498484 and NCT00002663 were approved by the Institutional Review Board of Memorial Sloan Kettering Cancer Center. Written consent was obtained from all patients prior to enrollment on trial.

### 7. INCORPORATION BY REFERENCE

All references cited herein are incorporated herein by reference in their entirety and for all purposes to the same extent as if each individual publication or patent or patent application was specifically and individually indicated to be incorporated by reference in its entirety for all purposes.

Many modifications and variations of this invention can be made without departing from its spirit and scope, as will be apparent to those skilled in the art. The specific embodiments described herein are offered by way of example only, and the invention is to be limited only by the terms of the appended claims, along with the full scope of equivalents to which such claims are entitled.

What is claimed is:

1. A method of selecting a T cell line from among a collection of T cell lines for therapeutic administration to a human patient to treat a disease or disorder associated with a pathogen or to treat a cancer in the human patient, wherein the human patient has not been the recipient of any cellular transplant, said method comprising: (a) identifying those T cell lines in the collection that exhibit a T cell response against one or more antigens of the pathogen or cancer and that are restricted by one or more HLA alleles shared with the human patient; (b) excluding from the T cell lines identified in step (a) those T cell lines that exhibit a T cell response against said one or more antigens of the pathogen or cancer and that are restricted by only one HLA allele shared with the human patient; and (c) selecting for therapeutic administration to said human patient a T cell line from among those identified T cell lines remaining after step (b).

2. The method of claim 1, which further comprises before step (a) a step of ascertaining the HLA restriction of each T cell line in the collection.

3. The method of claim 1 or 2, which further comprises before step (a) a step of ascertaining the HLA assignment of the human patient.

4. The method of claim 3, wherein the step of ascertaining the HLA assignment of the human patient comprises typing at least 4 HLA loci.

5. The method of any one of claims 1-4, wherein the selected T cell line is derived from a human donor that is allogeneic to the human patient.

6. A method of selecting a T cell line from among a collection of T cell lines for therapeutic administration to a human patient to treat a disease or disorder associated with a pathogen or to treat a cancer in the human patient, wherein the human patient has been the recipient of a cellular transplant, said method comprising: (a) identifying those T cell lines in the collection that exhibit a T cell response against one or more antigens of the pathogen or cancer and that are restricted by one or more HLA alleles shared with an entity selected from the group consisting of (i) the diseased cells in the human patient that express the one or more antigens of the pathogen or cancer, (ii) the human patient, (iii) the donor of the cellular transplant, and (iv) both the human patient and the donor of the cellular transplant; (b) excluding from the T cell lines identified in step (a) those T cell lines that exhibit a T cell response against said one or more antigens of the pathogen or cancer and that are restricted by only one HLA allele shared with the entity; and (c) selecting for therapeutic administration to said human patient a T cell line from among those identified T cell lines remaining after step (b).

7. The method of claim 6, which further comprises before step (a) a step of ascertaining the HLA restriction of each T cell line in the collection.

8. The method of claim 6 or 7, which further comprises before step (a) a step of ascertaining the HLA assignment of the entity.

9. The method of claim 8, wherein the step of ascertaining the HLA assignment of the entity comprises typing at least 4 HLA loci.

10. The method of any one of claims 6-9, wherein the selected T cell line is derived from a human donor that is allogeneic to the human patient.

11. The method of claim 10, wherein the human donor is a third-party donor that is different from the donor of the cellular transplant.

12. The method of any one of claims 6-11, wherein the cellular transplant is a hematopoietic stem cell transplant (HSCT).

13. The method of claim 12, wherein the disease or disorder or the cancer is an EBV-associated post-transplant lymphoproliferative disorder (EBV-PTLD) and the entity is the donor of the cellular transplant.

14. The method of any one of claims 6-11, wherein the cellular transplant is a solid organ transplant (SOT).

15. The method of claim 14, wherein the cellular transplant is a kidney transplant, a liver transplant, a heart transplant, an intestinal transplant, a pancreas transplant, a lung transplant, or a small bowel transplant.

16. The method of claim 14 or 15, wherein the disease or disorder or the cancer is an EBV-PTLD and the entity is the human patient.

17. The method of any one of claims 1-12 and 14-15, wherein the method is of selecting a T cell line for therapeutic administration to the human patient to treat a disease or disorder associated with a pathogen in the human patient, and the one or more antigens are one or more antigens of the pathogen.

18. The method of claim 17, wherein the pathogen is a virus, bacterium, fungus, helminth or protist.

19. The method of claim 18, wherein the pathogen is a virus.

20. The method of claim 19, wherein the virus is cytomegalovirus (CMV).

21. The method of claim 20, wherein the disease or disorder is CMV infection.

22. The method of claim 20 or 21, wherein the one or more antigens are CMV pp65, CMV IE1, or a combination thereof.

23. The method of claim 19, wherein the virus is Epstein-Barr virus (EBV).

24. The method of claim 23, wherein the one or more antigens are EBNA1, EBNA2, EBNA3A, EBNA3B, EBNA3C, LMP1, LMP2, or a combination thereof.

25. The method of claim 19, wherein the virus is BK virus (BKV), John Cunningham virus (JCV), human herpesvirus, human papillomavirus (HPV), hepatitis B virus (HBV), hepatitis C virus (HCV), herpes simplex virus (HSV), varicella zoster virus (VZV), Merkel cell polyomavirus (MCV), adenovirus (ADV), human immunodeficiency virus (HIV), influenza virus, ebola virus, poxvirus, rhabdovirus, or paramyxovirus.

26. The method of any one of claims 1-12 and 14-15, wherein the method is of selecting a T cell line for therapeutic administration to the human patient to treat a cancer in the human patient, and the one or more antigens are one or more antigens of the cancer.

27. The method of claim 26, wherein the cancer is a blood cancer.

28. The method of claim 26, wherein the cancer is a cancer of the breast, lung, ovary, stomach, pancreas, larynx, esophagus, testes, liver, parotid, biliary tract, colon, rectum, cervix, uterus, endometrium, kidney, bladder, prostate, thyroid, brain or skin.

29. The method of claim 26 wherein the one or more antigens is Wilms Tumor 1 (WT1).

30. The method of claim 29, wherein the cancer is multiple myeloma or plasma cell leukemia.

31. The method of claim 26, wherein the one or more antigens are one or more antigens of EBV.

32. The method of claim 31, wherein the cancer is an EBV-positive lymphoproliferative disorder.

33. The method of claim 26, wherein the one or more antigens are one or more antigens of CMV.

34. The method of claim 33, wherein the cancer is CMV-positive glioblastoma multiforme.
